(19) United States
(12) Patent Application Publication
(10) Pub. No.: US 2015/0231105 A1
(43) Pub. Date: Aug. 20, 2015

(54) METHOD FOR TREATING A CANCER CAUSED BY CANCER STEM CELLS

(71) Applicant: NEW BELLUS ENTERPRISES CO., LTD, Tainan City (TW)

(72) Inventors: Chun-Chih Huang, Tainan City (TW); Chih-Chieh Chen, Tainan City (TW); Lih-Geeng Chen, Tainan City (TW); Yew-Min Tzeng, Tainan City (TW); Chi-Tai Yeh, Tainan City (TW); Tsang-Hsien Alexander Wu, Tainan City (TW)

(73) Assignee: NEW BELLUS ENTERPRISES CO., LTD, Tainan City (TW)

(21) Appl. No.: 14/695,026

(22) Filed: Apr. 23, 2015

Related U.S. Application Data
(63) Continuation-in-part of application No. 13/649,984, filed on Oct. 11, 2012.

(30) Foreign Application Priority Data
Oct. 11, 2011 (TW) TW 100136825

(51) Int. Cl. A61K 31/22; A61K 31/555; A61K 31/513

(52) U.S. Cl. CPC A61K 31/22; A61K 31/513; A61K 31/555

(57) ABSTRACT

This invention provides a method for inhibiting tumor growth caused by cancer stem cells in a subject in need thereof, which comprises administering to the subject an effective amount of 4-acetyl-antroquinonol B.

### CROSS-REFERENCES TO RELATED APPLICATIONS

This application is a Continuation-in-Part application of the pending U.S. patent application Ser. No. 13/649,984 filed on Oct. 11, 2012, and claims priority to Taiwan Appl. No. TW 100136825, filed on Oct. 11, 2011, all of which are hereby incorporated by reference in their entireties.

### FIELD OF THE INVENTION

The present invention is related to a method for treating cancer, comprising administering an effective amount of Antrodia camphorata extracts to the subject.

### BACKGROUND OF THE INVENTION

### Cancer Stem Cells

The traditional cancer treatment is mainly to inhibit the growth of the fast proliferating cancer cells and to induce their apoptosis. However, because of the heterogeneity of cancer cells, cancer cells with high grades of malignancy often survive or escape from the detection of the immune system that leads to frequent recurrence of cancer after treatments of chemotherapy drugs and radiation therapy. In recent years, the rise of a new theory provides a new explanation for the reason why cancers are difficult to cure. Many studies indicate that the majority of cancer cells do not have the ability to cause tumor, only a very small portion of cancer cells are tumorigenic and can differentiate into a variety of cells in tumor tissue. Scientists found that these few cells in different cancer tissues, including leukemia, breast cancer, brain cancer, ovarian cancer, prostate cancer, colorectal cancer and oral cancer, have more resistance to radiation or drugs than other cancer cells. Therefore, these cancer cells with stem cell-like characteristics are named “cancer stem cells” (CSCs).

Current studies have indicated that cancer stem cells can be isolated from patients' tumor tissues. A handful of “cancer stem cells” can form a tumor in the patient's body. The regulation of activating and proliferating of such cancer stem cells is closely related to tumor recurrence, remote invasion and even the patient's survival rate. Similar to the normal stem cells, cancer stem cells also have the ability to self-renew and differentiate. They grow continuously and differentiate into tumor cells of different types and morphologies. However, unlike normal stem cells, the self-renewal mechanisms of cancer stem cells are not under normal regulation. Take the normal stem cell surface antigen CD133 for an example, CD133 is a glycoprotein having five transmembrane domains that was first identified from CD34⁺ precursor cells isolated from blood of adult, bone marrow and fetal liver cells and was regarded as a marker of hematopoietic stem cells. However, in the study of last five years, CD133 is regarded as a surface marker for the cancer stem cell of leukemia, brain cancer, retinoblastoma, kidney cancer, pancreatic cancer, prostate cancer and liver cancer. Recent reports also pointed out that there may be CD133⁺ cancer stem cells in medulloblastoma as well as glioma and the ability of proliferating and self-renewal of these CD133⁺ cells are better than the general tumor cells. Therefore, CD133 can be regarded as one of the important markers of cancer stem cells.

The discrimination of Cancer Stem Cells

According to the characteristics of cancer stem cells, three ways are provided to successfully isolate cancer stem cells from solid tumors. First, based on the specific surface antigen expressed on the cancer stem cells, such as CD44 or of CD133, flow cytometry was used to isolate the cancer stem cells. CD133 was isolated from the cancer stem cells of a variety of brain tumors, including glioblastoma multiforme, children medulloblastoma and ependymomas. In addition, it was also found in the cancer stem cells of colon cancer. There are approximately 1.8-24.5% of cells expressing CD133 in colon cancer and most of these cells have the ability to form tumors. Second, the fluorescent dye Hoechst 33342 was used to stain tumor tissues or cancer cell groups and then the side population without fluorescent signals, which are cancer stem cells, was isolated. These cells may lead to tumor chemoresistance. The expression of ABCG2, an ATPase transporter, is closely related to the side population phenomenon. Because of the high expression of ABCG2 on cell membrane of stem cells, the transporter will actively pump Hoechst 33342 from inside of the nucleus to outside. These side populations of cells analyzed by flow cytometry were defined as cells with the characteristic of stem cells. However, the latest studies have shown that the cancer cells have similar tumorigenicity regardless of their expression of ABCG2. Third, the tumor tissue or cancer cells were cultured in medium that is serum-free but containing specific growth factors, such as basic fibroblast growth factor (bFGF), epidermal growth factor (EGF) and other synthetic growth factors. The sphere body formation cells are abundant in cancer stem cells. It is assumed that the serum-free culture environment can help the cancer stem cells maintain in the undifferentiated state.

Molecular markers are mainly used to confirm the cell surface antigen or specific transcription factors. Various types of stem cells and cancer stem cells need to be confirmed by using different markers and then detecting the ability of self-renewal and differentiation of the stem cells. Therefore, the primary goal of the top research teams in various countries is to search for the surface markers or specific gene cluster unique and specific to cancer stem cells and identify the cancer stem cells with tumorigenic ability. In addition, if the cancer stem cells can be correctly and successfully isolated, the basic research of the subsequent gene regulation, human body repair or the development of drug screening platform or the direct application of individualized anti-cancer treatment in cancer patients can be conducted by in vitroculture.

### Chemoresistance & Radioresistance

Recently, the kinds of cancer stem cells are regarded as cells having the potential to develop into cancers. In the experimental animal model, it is also proved that a very small amount of cancer stem cells is enough to form tumors. However, other non cancer stem cells need much greater number of cells to achieve similar tumorigenicity. On the other hand, because of the proliferating rate of the cancer stem cells are extremely slow, even in the non-dividing state, and the expressing amount of ABC transport proteins is far more than the normal cancer cells, the cancer stem cells cannot be killed easily by chemotherapy drugs or radiation. Thus, the cancer stem cells are not only the main reason for cancer recurrence after treatment and the ineffectiveness of drugs but also the main reason for malignant cancer metastasis. This shows that the opportunity to cure cancers is to eliminate the cancer stem cells. Therefore, one of the important topics in recent cancer research is to identify the difference between such cells and the normal cancer cells or normal stem cells, to develop effective strategy for killing cancer stem cells specific to such features .

In recent years, the medical profession proposes a new vision for such difficult-to-cure and easy-to-relapse and metastasis situation. The specific “cancer stem cell” may exist in different cancer tissues. It is the key point to cause tumor recurrence in cancer patients. The traditional cancer treatments are radiation and chemotherapy after surgery to inhibit the growth of cancer cells and to induce their apoptosis. However, the cancer cells with high malignancy can survive from chemotherapy drugs and radiation treatment and escape from the detection of the immune system that are easily recurrent after treatments.

### Antrodia Camphorate

Antrodia camphorata is also called Niu Chang-Zhi, Niu Chang-Gu, red camphor mushroom and the like, which is a perennial mushroom belonging to the order Aphyllophorales, the family Polyporaceae. It is an endemic species in Taiwan growing on the inner rotten heart wood wall of Cinnamomum kanehirae Hay. Cinnamoum kanehirai Hay is rarely distributed and being overcut unlawfully, which makes Antrodia camphorata growing inside the tree in the wild became even rare. The price of Antrodia camphorata is very expensive due to the extremely slow growth rate of natural Antrodia camphorata that only grows between June to October.

In traditional Taiwanese medicine, Antrodia camphorata is commonly used as an antidotal, liver protective, anti-cancer drug. Antrodia camphorata, like general edible and medicinal mushrooms, is rich in numerous nutrients including triterpenoids, polysaccharides (such as [beta]-glucosan), adenosine, vitamins (such as vitamin B, nicotinic acid), proteins (immunoglobulins), superoxide dismutase (SOD), trace elements (such as calcium, phosphorus and germanium and so on), nucleic acid, steroids, and stabilizers for blood pressure (such as antodia acid) and the like. These physiologically active ingredients are believed to exhibit effects such as: anti-tumor activities, increasing immuno-modulating activities, anti-allergy, anti-bacteria, anti-high blood pressure, decreasing blood sugar, decreasing cholesterol, and the like. Now there are only researches for the inhibitory effects of Antrodia camphorata for normal cancer cells, but no researches for the cancer stem cells.

### BRIEF DESCRIPTION OF THE DRAWINGS

FIG. 1 shows the producing process of A. camphorata extracts used in the present invention.

FIG. 2 shows the ESI(+)-MS Mass spectrum of 4-acetyl-antroquinonol B.

FIG. 3 shows the UV absorption spectra of 4-acetyl-antroquinonol B.

FIG. 4 shows the ¹H-NMR (500 MHz, CDCl₃) spectra of 4-acetyl-antroquinonol B.

FIG. 5 shows the ¹³C-NMR (125 MHz, CDCl₃) spectra of 4-acetyl-antroquinonol B.

FIG. 6 shows the pH of A. camphorata concentrates and the FIG. for inhibiting the growth of human stem cells.

FIG. 7 shows the cytotoxicity curve of A. camphorata concentrate (D1) for inhibiting human Lung cancer stem cell (Lung CSC).

FIG. 8 shows the cytotoxicity curve of ethyl acetate extracts of A. camphorata concentrate (D2) for inhibiting human Lung cancer stem cell (Lung CSC).

FIG. 9 shows the cytotoxicity curve of lyophilized powder of A. camphorata concentrate (D3) for inhibiting human Lung cancer stem cell (Lung CSC).

FIG. 10 shows the cytotoxicity curve of ethyl acetate extracts of lyophilized powder of A. camphorata concentrate (D4) for inhibiting human Lung cancer stem cell (Lung CSC).

FIG. 11 shows the cytotoxicity curve of ethyl acetate extracts of A. camphorata mycelium (D5) for inhibiting human Lung cancer stem cell (Lung CSC).

FIG. 12 shows the cytotoxicity curve of A. camphorata concentrate (D1) for inhibiting human fibroblast AF-1(Adult fibroblast-1).

FIG. 13 shows the cytotoxicity curve of ethyl acetate extracts of A. camphorata concentrate (D2) for inhibiting human fibroblast AF-1(Adult fibroblast-1).

FIG. 14 shows the cytotoxicity curve of lyophilized powder of A. camphorata concentrate (D3) for inhibiting human fibroblast AF-1(Adult fibroblast-1).

FIG. 15 shows the cytotoxicity curve of ethyl acetate extracts of lyophilized powder of A. camphorata concentrate (D4) for inhibiting human fibroblast AF-1(Adult fibroblast-1).

FIG. 16 shows the cytotoxicity curve of ethyl acetate extracts of A. camphorata mycelium (D5) for inhibiting human fibroblast AF-1(Adult fibroblast-1).

FIG. 17 shows the cytotoxicity curve of A. camphorata concentrate (D1) for inhibiting Glioblastomas multiform cancer stem cells (GBM CSC).

FIG. 18 shows the cytotoxicity curve of ethyl acetate extracts of A. camphorata concentrate (D2) for inhibiting Glioblastomas multiform cancer stem cells (GBM CSC).

FIG. 19 shows the cytotoxicity curve of lyophilized powder of A. camphorata concentrate (D3) for inhibiting Glioblastomas multiform cancer stem cells (GBM CSC).

FIG. 20 shows the cytotoxicity curve of ethyl acetate extracts of lyophilized powder of A. camphorata concentrate (D4) for inhibiting Glioblastomas multiform cancer stem cells (GBM CSC).

FIG. 21 shows the cytotoxicity curve of ethyl acetate extracts of A. camphorata mycelium (D5) for inhibiting Glioblastomas multiform cancer stem cells (GBM CSC).

FIG. 22 shows the cytotoxicity curve of A. camphorata concentrate (D1) for inhibiting human fibroblast AF-2(Adult fibroblast-2).

FIG. 23 shows the cytotoxicity curve of ethyl acetate extracts of A. camphorata concentrate (D2) for inhibiting human fibroblast AF-2(Adult fibroblast-2).

FIG. 24 shows the cytotoxicity curve of lyophilized powder of A. camphorata concentrate (D3) for inhibiting human fibroblast AF-2(Adult fibroblast-2).

FIG. 25 shows the cytotoxicity curve of ethyl acetate extracts of lyophilized powder of A. camphorata concentrate (D4) for inhibiting human fibroblast AF-2(Adult fibroblast-2).

FIG. 26 shows the cytotoxicity curve of ethyl acetate extracts of A. camphorata mycelium (D5) for inhibiting human fibroblast AF-2(Adult fibroblast-2).

FIG. 27 shows the cytotoxicity curve of A. camphorata concentrate (D1) for inhibiting Head and neck squamous cell carcinoma cancer stem cells (HNSCC CSC).

FIG. 28 shows the cytotoxicity curve of ethyl acetate extracts of A. camphorata concentrate (D2) for inhibiting Head and neck squamous cell carcinoma cancer stem cells (HNSCC CSC).

FIG. 29 shows the cytotoxicity curve of lyophilized powder of A. camphorata concentrate (D3) for inhibiting Head and neck squamous cell carcinoma cancer stem cells (HNSCC CSC).

FIG. 30 shows the cytotoxicity curve of ethyl acetate extracts of lyophilized powder of A. camphorata concentrate (D4) for inhibiting Head and neck squamous cell carcinoma cancer stem cells (HNSCC CSC).

FIG. 31 shows the cytotoxicity curve of ethyl acetate extracts of A. camphorata mycelium (D5) for inhibiting Head and neck squamous cell carcinoma cancer stem cells (HNSCC CSC).

FIG. 32 shows the cytotoxicity curve of A. camphorata concentrate (D1) or inhibiting colorectal cancer stem cells (CRC CSC).

FIG. 33 shows the cytotoxicity curve of ethyl acetate extracts of A. camphorata concentrate (D2) for inhibiting colorectal cancer stem cells (CRC CSC).

FIG. 34 shows the cytotoxicity curve of lyophilized powder of A. camphorata concentrate (D3) for inhibiting colorectal cancer stem cells (CRC CSC).

FIG. 35 shows the cytotoxicity curve of ethyl acetate extracts of lyophilized powder of A. camphorata concentrate (D4) for inhibiting colorectal cancer stem cells (CRC CSC).

FIG. 36 shows the cytotoxicity curve of ethyl acetate extracts of A. camphorata mycelium (D5) for inhibiting colorectal cancer stem cells (CRC CSC).

FIG. 37 shows the cytotoxicity curve of A. camphorata concentrate (D1) for inhibiting breast cancer stem cells (Breast CSC).

FIG. 38 shows the cytotoxicity curve of ethyl acetate extracts of A. camphorata concentrate (D2) for inhibiting breast cancer stem cells (Breast CSC).

FIG. 39 shows the cytotoxicity curve of lyophilized powder of A. camphorata concentrate (D3) for inhibiting breast cancer stem cells (Breast CSC).

FIG. 40 shows the cytotoxicity curve of ethyl acetate extracts of lyophilized powder of A. camphorata concentrate (D4) for inhibiting breast cancer stem cells (Breast CSC).

FIG. 41 shows the cytotoxicity curve of ethyl acetate extracts of A. camphorata mycelium (D5) for inhibiting breast cancer stem cells (Breast CSC).

FIG. 42 shows the cytotoxicity curve of ethyl acetate extracts of lyophilized powder of A. camphorata concentrate (D4) for inhibiting hepatoma cancer stem cells (Hepatoma CSC).

FIG. 43 shows the cytotoxicity curve of ethyl acetate extracts of A. camphorata mycelium for inhibiting hepatoma cancer stem cells (Hepatoma CSC).

FIG. 44 shows the cytotoxicity curve of ethyl acetate extracts of lyophilized powder of A. camphorata concentrate (D4) for inhibiting leukemia cancer stem cells (Leukemia CSC).

FIG. 45 shows the cytotoxicity curve of ethyl acetate extracts of A. camphorata mycelium (D5) for inhibiting leukemia cancer stem cells (Leukemia CSC).

FIG. 46 shows the cytotoxicity curve of ethyl acetate extracts of lyophilized powder of A. camphorata concentrate (D4) for inhibiting gastric cancer stem cells (Gastric CSC).

FIG. 47 shows the cytotoxicity curve of ethyl acetate extracts of A. camphorata mycelium (D5) for inhibiting gastric cancer stem cells (Gastric CSC).

FIG. 48 shows the effect of the combination of Ionizing Radiation treatment (2 Gy) for Glioblastomas multiform cancer stem cells (GBM CSC).

FIG. 49 shows the effect of the combination of Ionizing Radiation treatment (2 Gy) for lung cancer stem cells (Lung CSC).

FIG. 50 shows the effect of the combination of Ionizing Radiation treatment (2 Gy) for Head and neck squamous cell carcinoma cancer stem cells (HNSCC CSC).

FIG. 51 shows the effect of the combination of Ionizing Radiation treatment (2 Gy) for breast cancer stem cells (Breast CSC).

FIG. 52 shows the effect of the combination of Ionizing Radiation treatment (2 Gy) for hepatoma cancer stem cells (Hepatoma CSC).

FIG. 53 shows the effect of the combination of Ionizing Radiation treatment (2 Gy) for colorectal cancer stem cells (CRC CSC).

FIG. 54 shows the effect of the combination of Ionizing Radiation treatment (4 Gy) for Glioblastomas multiform cancer stem cells (GBM CSC).

FIG. 55 shows the effect of the combination of Ionizing Radiation treatment (4 Gy) for lung cancer stem cells (Lung CSC).

FIG. 56 shows the effect of the combination of Ionizing Radiation treatment (4 Gy) for Head and neck squamous cell carcinoma cancer stem cells (HNSCC CSC).

FIG. 57 shows the effect of the combination of Ionizing Radiation treatment (4 Gy) for breast cancer stem cells (Breast CSC).

FIG. 58 shows the effect of the combination of Ionizing Radiation treatment (4 Gy) for hepatoma cancer stem cells (Hepatoma CSC).

FIG. 59 shows the effect of the combination of Ionizing Radiation treatment (4 Gy) for colorectal cancer stem cells (CRC CSC).

FIG. 60 shows the effect of the combination of chemotherapy drugs treatment (Cisplatin, 10 μg/ml) for Glioblastomas multiform cancer stem cells (GBM CSC).

FIG. 61 shows the effect of the combination of chemotherapy drugs treatment (Cisplatin, 10 μg/ml) for lung cancer stem cells (Lung CSC).

FIG. 62 shows the effect of the combination of chemotherapy drugs treatment (Cisplatin, 10 μg/ml) for Head and neck squamous cell carcinoma cancer stem cells (HNSCC CSC).

FIG. 63 shows the effect of the combination of chemotherapy drugs treatment (Cisplatin, 10 μg/ml) for breast cancer stem cells (Breast CSC).

FIG. 64 shows the effect of the combination of chemotherapy drugs treatment (Cisplatin, 10 μg/ml) for hepatoma cancer stem cells (Hepatoma CSC).

FIG. 65 shows the effect of the combination of chemotherapy drugs treatment (Cisplatin, 10 μg/ml) for colorectal cancer stem cells (CRC CSC).

FIG. 66 shows the effect of the combination of chemotherapy drugs treatment (Taxol, 5 ng/ml) for Glioblastomas multiform cancer stem cells (GBM CSC).

FIG. 67 shows the effect of the combination of chemotherapy drugs treatment (Taxol, 5 ng/ml) for lung cancer stem cells (Lung CSC).

FIG. 68 shows the effect of the combination of chemotherapy drugs treatment (Taxol, 5 ng/ml) for Head and neck squamous cell carcinoma cancer stem cells (HNSCC CSC).

FIG. 69 shows the effect of the combination of chemotherapy drugs treatment (Taxol, 5 ng/ml) for breast cancer stem cells (Breast CSC).

FIG. 70 shows the effect of the combination of chemotherapy drugs treatment (Taxol, 5 ng/ml) for hepatoma cancer stem cells (Hepatoma CSC).

FIG. 71 shows the effect of the combination of chemotherapy drugs treatment (Taxol, 5 ng/ml) for colorectal cancer stem cells (CRC CSC).

FIG. 72 shows the cytotoxicity curve of 4-acetyl-antroquinonol B for inhibiting Lung adenocarcinoma CD133 positive cancer stem cells.

FIG. 73 shows the cytotoxicity curve of 4-acetyl-antroquinonol B for inhibiting oral cancer stem cells (Oral CSC).

FIG. 74 shows the cytotoxicity curve of 4-acetyl-antroquinonol B for inhibiting Glioblastomas multiform cancer stem cells (GBM CSC).

FIG. 75 shows the cytotoxicity curve of 4-acetyl-antroquinonol B for inhibiting breast cancer stem cells (Breast CSC).

FIG. 76 shows the cytotoxicity curve of 4-acetyl-antroquinonol B for inhibiting lung cancer stem cells (Lung CSC).

FIG. 77 shows the cytotoxicity curve of 4-acetyl-antroquinonol B for inhibiting colorectal cancer stem cells (CRC CSC).

FIGS. 78A-78C show in vivo comparative analysis of the anti-tumorigenic effects among 4-AAQB alone and different regimens.

FIG. 79 shows the quantitative analysis of anti-tumor effects by 4-AAQB and other regimens.

FIG. 80 shows the comparative body weight analysis among different treatment groups.

### SUMMARY OF THE INVENTION

The present invention provides a method for inhibiting tumor growth caused by cancer stem cells in a subject in need thereof, which comprises administering to the subject an effective amount of 4-acetyl-antroquinonol B.

### DETAIL DESCRIPTION OF THE INVENTION

To assess the potential for inhibiting cancer stem cells for lung cancer, brain tumor, head and neck cancer, colorectal cancer and breast cancer, a total of five kinds of A. camphorata extracts were prepared as follows: A. camphorata concentrate (D1), ethyl acetate extracts of A. camphorata concentrate (D2), lyophilized powder of A. camphorata concentrate (D3), ethyl acetate extracts of lyophilized powder of A. camphorata concentrate (D4) and ethyl acetate extracts of A. camphorata mycelium (D5).

The purpose of the present invention is to screen for the A. camphorata extracts with anti-cancer activity. MTT (3-(4,5-dimethylthiazol-2-yl)-2,5-diphenyltetrazolium bromide) assay was used to complete the cytotoxicity test. Human lung cancer stem cells (lung CSC), human Adult fibroblast-1 (AF-1), glioblastoma multiforme cancer stem cells (GBM CSC), human Adult fibroblast-2 (AF-2), head and neck squamous cell carcinoma cancer stem cells (HNSCC CSC), colorectal cancer stem cells (CRC CSC), as well as breast cancer stem cells (Breast CSC), hepatoma cancer stem cells (hepatoma CSC), leukemia cancer stem cells (leukemia CSC) and gastric cancer stem cells (Gastric CSC) are used in cell experiments of the present invention.

The present invention provides a method for treating cancer caused by cancer stem cells in a subject in need thereof, which comprises administering to the subject an effective amount of Antrodia camphorata extracts. The Antrodia camphorata extracts are selected from the group consisting of ethyl acetate extracts of lyophilized powder of Antrodia camphorata concentrate and ethyl acetate extracts of Antrodia camphorata mycelium. The cancer stem cells are selected from liver cancer stem cells, lung cancer stem cells, brain tumor stem cells, head and neck cancer stem cells, colorectal cancer stem cells, breast cancer stem cells, leukemia cancer stem cells or gastric cancer stem cells. In a more preferred embodiment, the liver cancer stem cells are hepatoma cancer stem cells; the brain tumor stem cells are glioblastoma multiforme cancer stem cells; the head and neck cancer stem cells are head and neck squamous cell carcinoma cancer stem cells.

The effective amount of the Antrodia camphorata extracts ranges from 10 μg/ml to 500 μg/ml. In a more preferred embodiment, the effective amount of the Antrodia camphorata extracts ranges from 20 μg/ml to 400 μg/ml. In a most preferred embodiment, the effective amount of the Antrodia camphorata extracts ranges from 40 μg/ml to 300 μg/ml.

The method further comprises co-administration of a chemotherapy drug to increase inhibitory effect of the cancer stem cells. The chemotherapy drug is selected from Cisplatin or Taxol.

The method further comprises co-administration of ionizing radiation to increase inhibitory effect of the cancer stem cells.

The present invention also provides a method for treating cancer caused by cancer stem cells in a subject in need thereof, which comprises administering to the subject an effective amount of 4-acetyl-antroquinonol B. The cancer stem cells are selected from lung adenocarcinoma CD133 positive cancer stem cells, lung cancer stem cells, brain tumor stem cells, breast cancer stem cells, oral cancer stem cells or colorectal cancer stem cells. In a more preferred embodiment, the brain tumor stem cells are glioblastoma multiforme cancer stem cells.

The effective amount of the 4-acetyl-antroquinonol B ranges from 0.1 μg/ml to 100 μg/ml. In a more preferred embodiment, the effective amount of the 4-acetyl-antroquinonol B ranges from 1 μg/ml to 80 μg/ml. In a most preferred embodiment, the effective amount of the 4-acetyl-antroquinonol B ranges from 5 μg/ml to 60 μg/ml.

The present invention further provides a method for inhibiting tumor growth caused by cancer stem cells in a subject in need thereof, which comprises administering to the subject an effective amount of 4-acetyl-antroquinonol B. The cancer stem cells are selected from lung adenocarcinoma CD133 positive cancer stem cells, lung cancer stem cells, brain tumor stem cells, breast cancer stem cells, oral cancer stem cells, or colorectal cancer stem cells. Preferably, the cancer stem cells are colorectal cancer stem cells. The 4-acetyl-antroquinonol B can be administered orally or by injection. The subject includes but not limited to human and rodent. In a preferred embodiment, the effective amount ranges from about 2.5 mg/kg to about 5 mg/kg for intraperitoneal injection in rodents.

It is noted that the effective amount described above is for using in mice by intraperitoneal injection. Therefore, if the subject in need of such treatment is a human, then the amount should be recalculated according to known methods in the art. Also, the effective amount can be adapted for different administration routes according to known methods in the art.

For example, if the 4-acetyl-antroquinonol B is administered by oral administration, then the effective amount for using in mice orally will range from about 5 mg/kg to about 10 mg/kg.

The conversion of the dosage between a mouse and a human can refer to the following formula:

Human effective dose (HED) (in mg/kg)=Animal Dose (mg/kg)×[Animal Kₘ/Human Kₘ]
(Human K_{m=37;} Mouse Kₘ₌₃)

That is, the effective amount 5-10 mg/kg for using in mice by oral administration is approximately equal to about 0.4-0.8 mg/kg for using in humans by oral administration.

Therefore, in another embodiment, the effective amount ranges from about 0.4 mg/kg to about 0.8 mg/kg for oral administration in humans, and from about 0.2 mg/kg to about 0.4 mg/kg for injection in humans.

According to the above, a reasonable effective amount of 4-acetyl-antroquinonol B may range from about 0.1 mg/kg to about 10 mg/kg, or from about 0.2 mg/kg to about 5 mg/kg. An appropriate dose expansion in the upper or lower limit can also be expected to retain a similar efficacy.

The above method also comprises administering to the subject a combination of 4-acetyl-antroquinonol B, Fluorouracil, and Oxaliplatin. The 4-acetyl-antroquinonol B in this combination plays a role of mitigating body weight loss caused by administration of Fluorouracil and Oxaliplatin.

### EXAMPLES

### Example 1

Preparation of A. camphorata Extracts

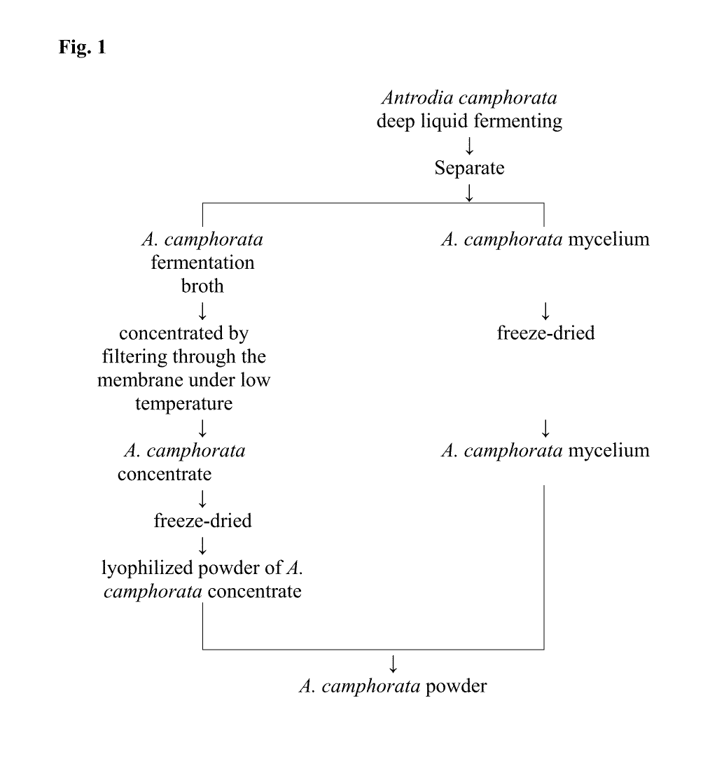

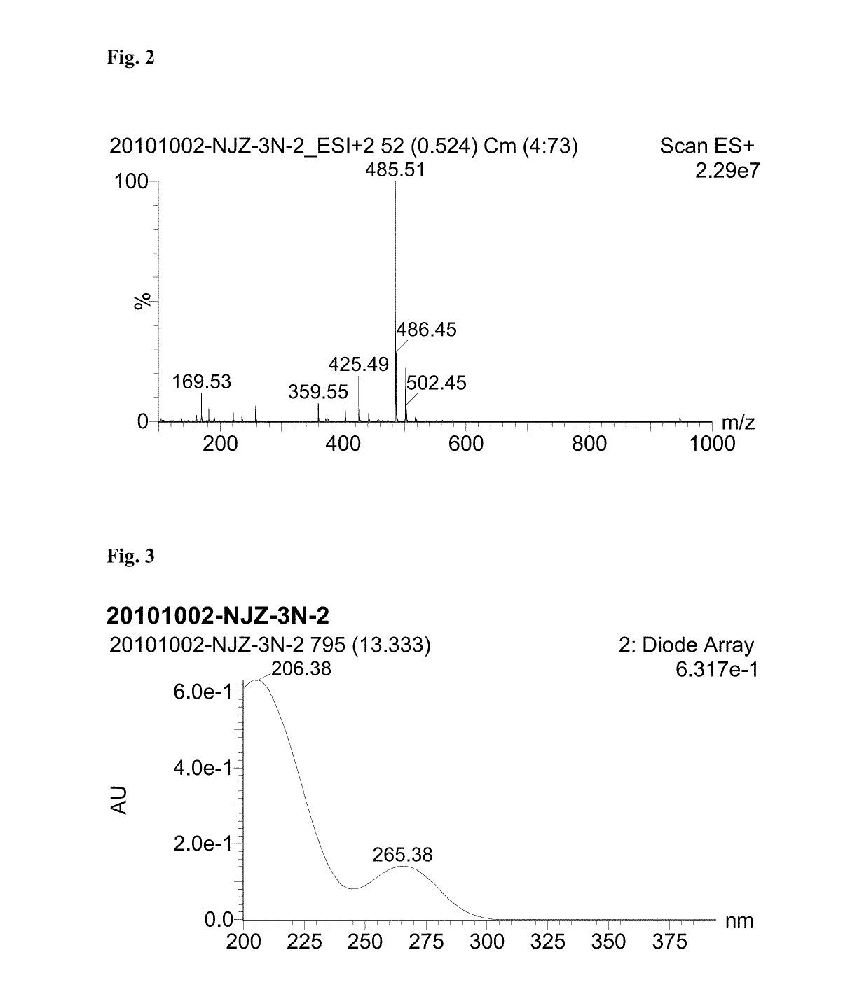

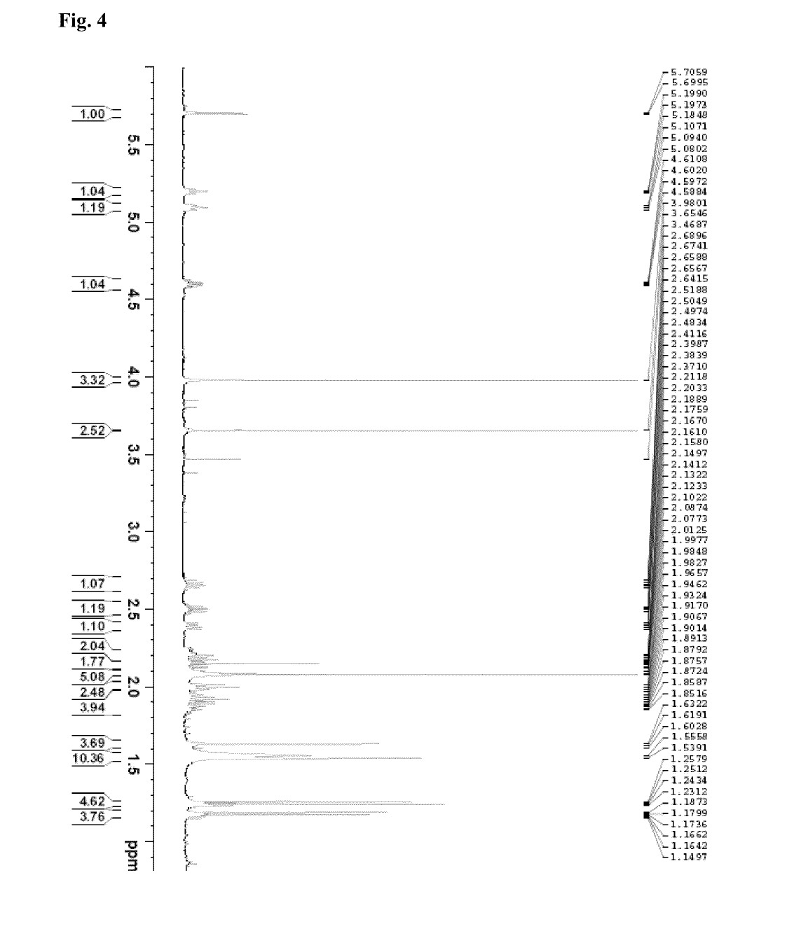

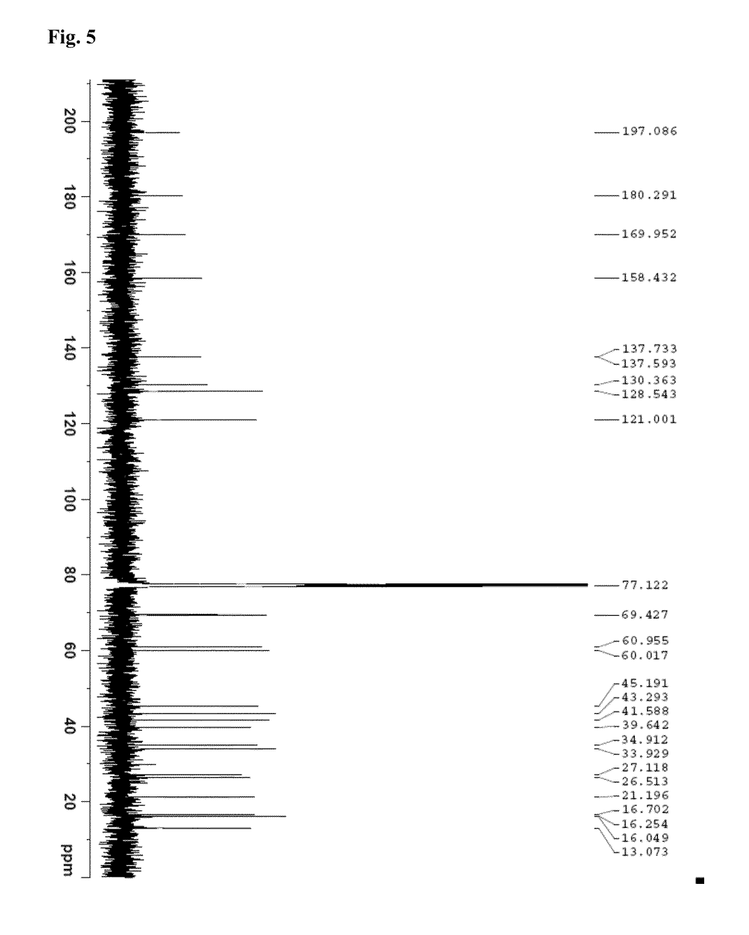

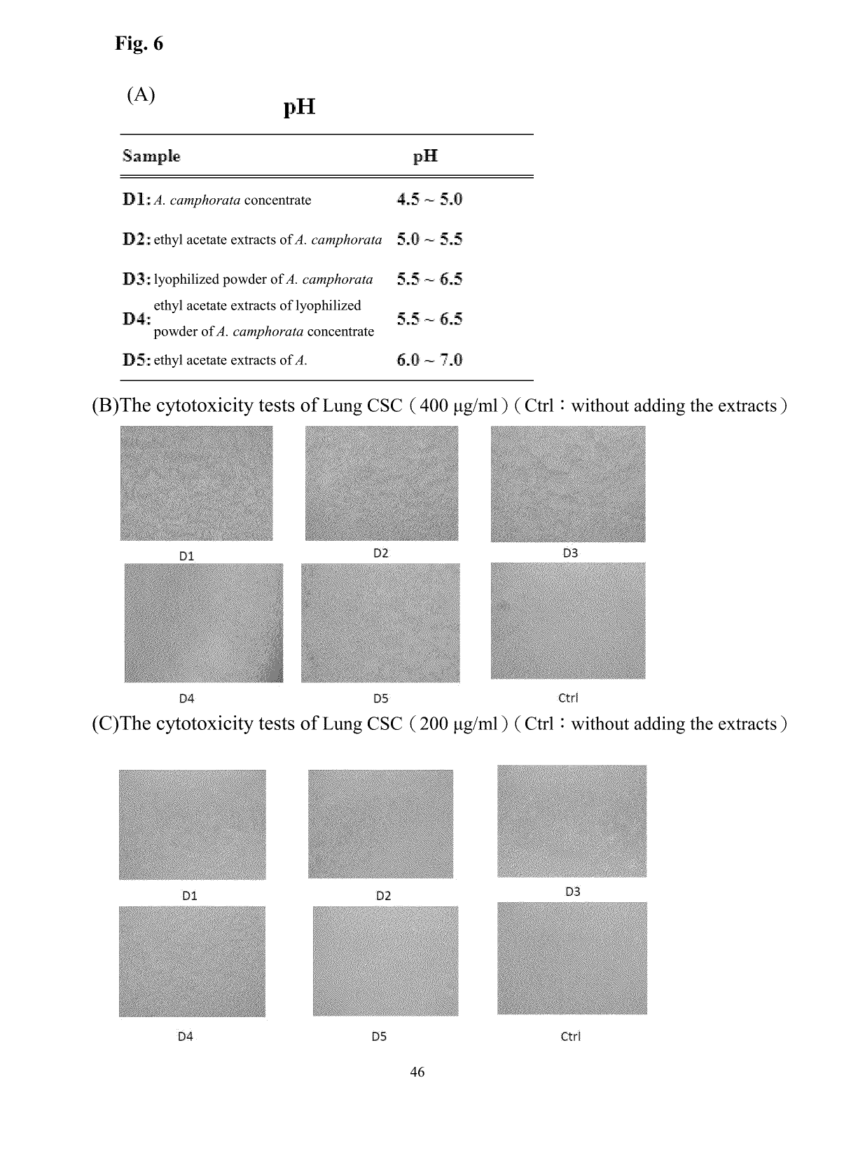

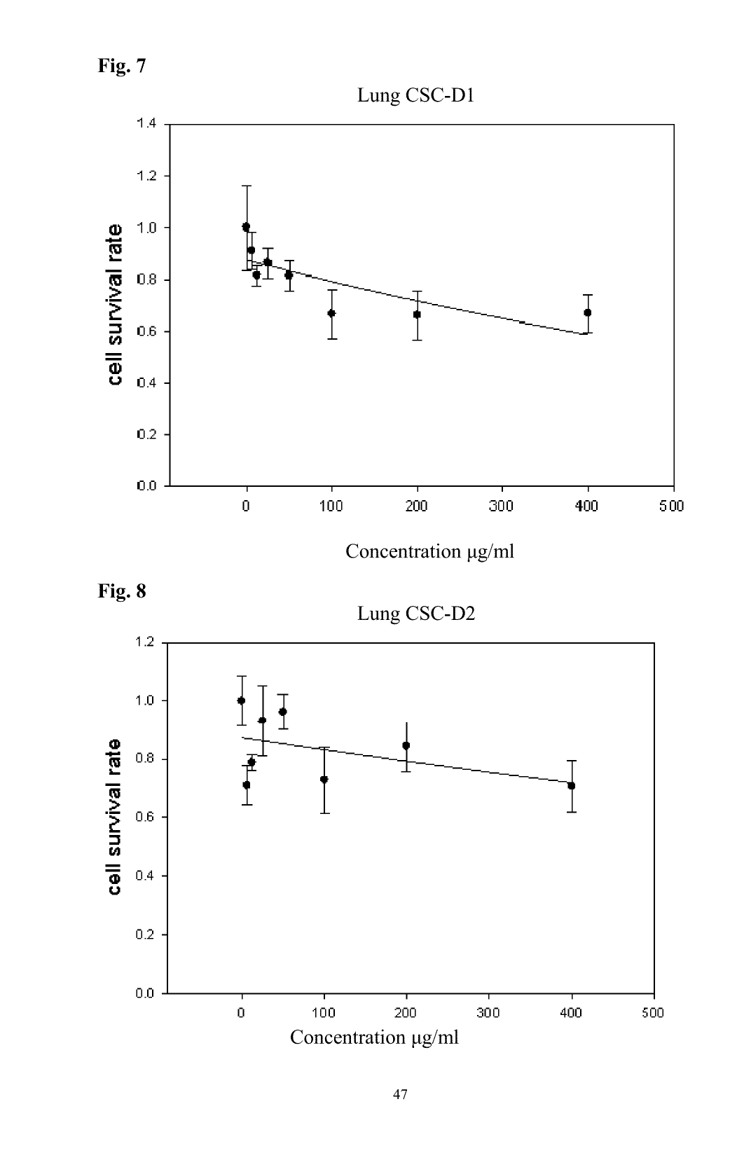

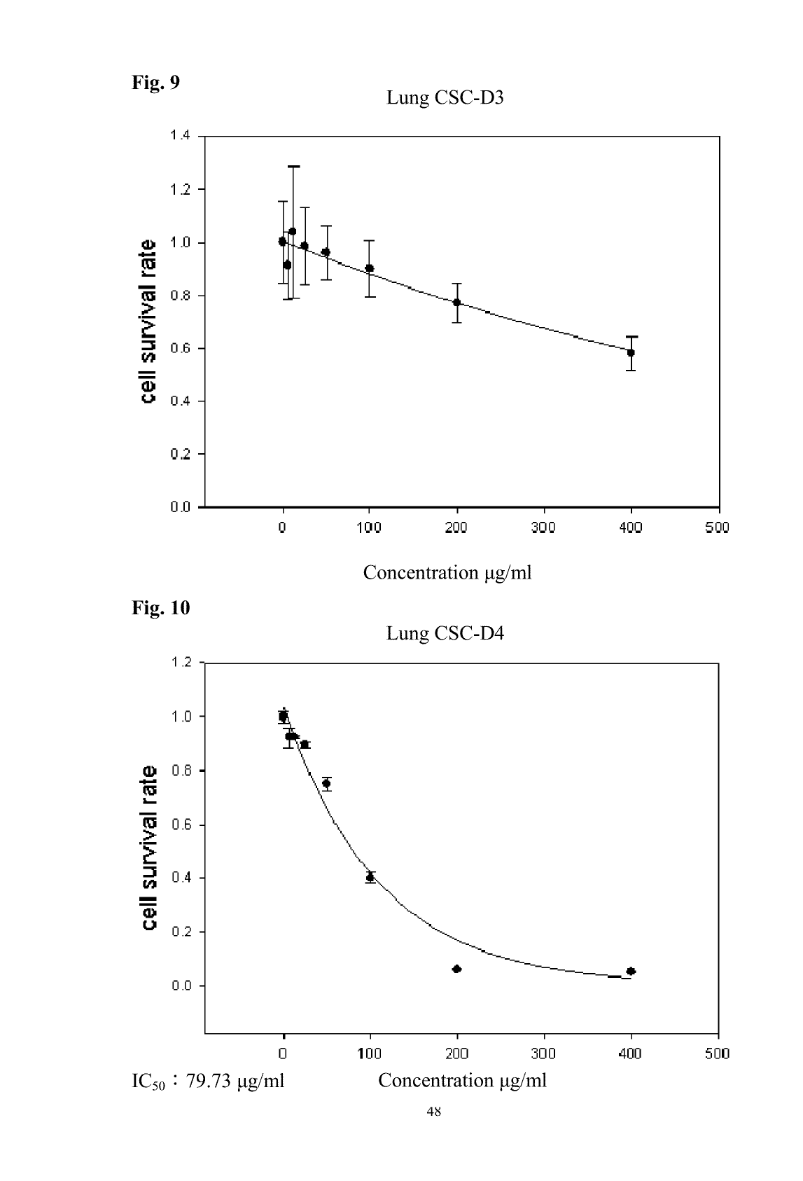

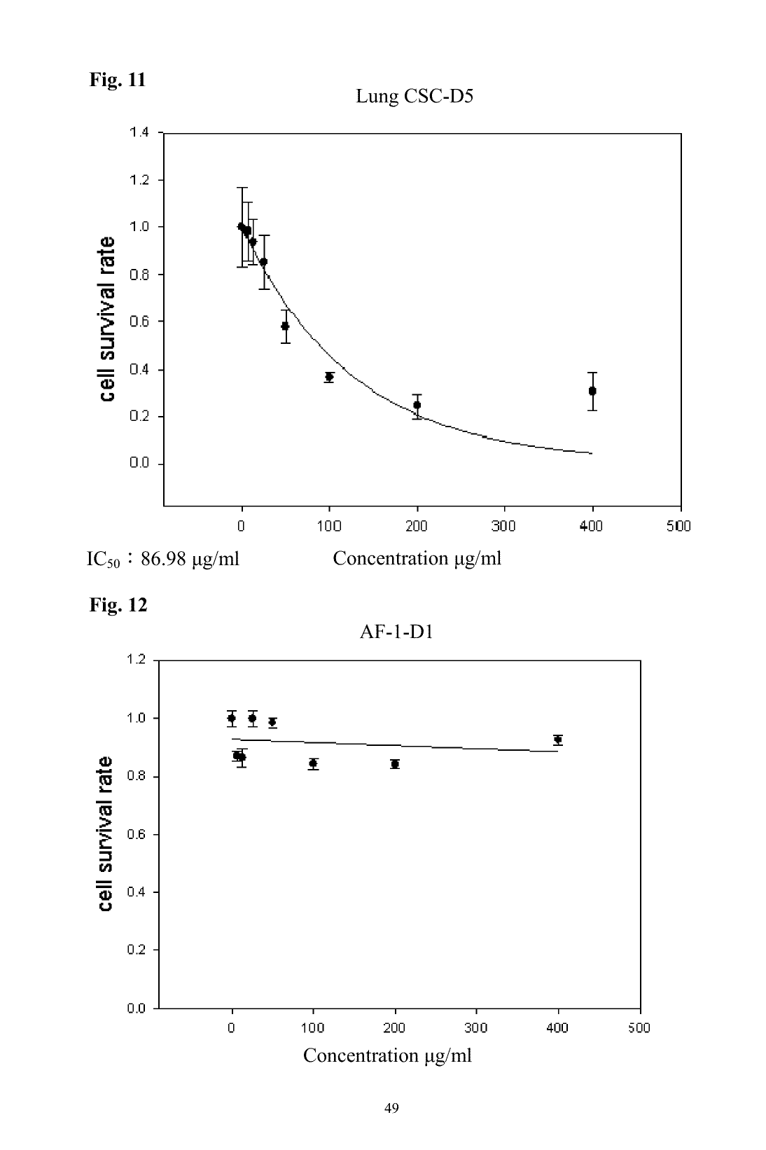

The A. camphorata was incubated to produce A. camphorata fermented concentrate. The production process of A. camphorata was shown in FIG. 1 and a total of five A. camphorata extracts were prepared as follows:
1. A. camphorata concentrate (D1),
2. ethyl acetate extracts of A. camphorata concentrate (D2);
3. lyophilized powder of A. camphorata concentrate (D3);
4. ethyl acetate extracts of lyophilized powder of A. camphorata concentrate (D4);
5. ethyl acetate extracts of A. camphorata mycelium (D5)

The A. camphorata was incubated to generate A. camphorata fermentation broth and then was concentrated by filtering through the membrane under low temperature to generate A. camphorata concentrate (D1) and the A. camphorata concentrate were further freeze-dried to generate the lyophilized powder of A. camphorata concentrate (D3).

Preparation of ethyl acetate extracts of A. camphorata concentrate (D2): 100 ml of A. camphorata concentrate was added into 100 ml of ethyl acetate to partition (3 times). The ethyl acetate layer was collected, concentrated and dried to generate the ethyl acetate extracts.

Preparation of ethyl acetate extracts of lyophilized powder of A. camphorata concentrate (D4): 10 g of lyophilized powder of A. camphorata concentrate was added into 100 ml of 95% ethanol for reflux extraction for 3 hours. After sample was concentrated and dried, 100 ml of water and 100 ml of ethyl acetate were added to partition (3 times). The ethyl acetate layer was collected, concentrated and dried to generate the ethyl acetate extracts of lyophilized powder of A. camphorata concentrate.

Preparation of the ethyl acetate extracts of A. camphorata mycelium (D5): 10 g of A. camphorata mycelium was added into 100 ml of 95% ethanol for reflux extraction for 3 hours. After sample was concentrated and dried, 100 ml of water and 100 ml of ethyl acetate were added to partition (3 times). The ethyl acetate layer was collected, concentrated and dried to generate the ethyl acetate extracts of A. camphorata mycelium.

Isolation of the 4-acetyl-antroquinonol B of the ethyl acetate Extracts of A. Camphorata mycelium (D5)

3 kg of A. camphorata mycelium was added to 10 L of 95% of ethanol and heated for reflux extraction for 4 times. The extract was filtered and concentrated, then dried under reduced pressure to generate 384 g of ethanol extracts. The ethanol extracts was suspended with water and partitioned with equal amount of ethyl acetate. The ethyl acetate layer was concentrated under reduced pressure to obtain 157.57 g of ethyl acetate layer partition and 159.51 g of water layer partition .

The 157.57 g of ethyl acetate layer partition was chromatographed on silica gel column (10 cm i.d×30 cm). Following the order of n-hexane→n-hexane-ethyl acetate (10:1→10:2→10:3→10:4→10:5→1:1→1:2, v/v)→ethyl acetate→methanol, 10 L of each proportion were used to elute and each 1 L was collected as a partition. The eluted partition 56-63(3.015 g) of n-hexane-ethyl acetate (10:4) was chromatographed using reversed phase preparative column (Tosoh ODS-80Ts, 21.5 mm×300 mm, 10 μm). H₂O—CH₃CN (20:80) was used as the mobile phase at a flow rate of 10 ml/min for chromatography, and the detecting wavelength was 265 nm, the column temperature was fixed at 40° C. 4-acetyl-antroquinonol B (131 mg) was obtained.

Identification of the Structure of 4-acetyl-antroquinonol B

4-acetyl-antroquinonol B, ESI(+)MS (m/z): 485 [M+Na]^{+, 502} [M+K]⁺. UV λₘₐₓ (nm): 206, 265. ¹H-NMR (500 MHz, CDCl₃) δ (ppm): 5.70 (1H, d, J=3.2 Hz, H-4), 5.20 (1H, t, J=6.4 Hz, H-12), 5.09 (1H, t, J=6.8 Hz, H-8), 4.60 (1H, m, H-15), 3.98 (3H, s, H-24), 3.65 (3H, s, H-23), 2.67 (1H, m, H-17), 2.50 (1H, dq, J=6.9, 10.8 Hz, H-6), 2.39 (1H, dd, J=6.5, 13.9 Hz, H-14), 2.23 (1H, m, H-11), 2.19 (1H, m, H-14), 2.14 (1H, m, H-16), 2.09 (2H, m, H-7), 2.08 (3H, OAc), 2.00 (2H, m, H-10), 1.94 (1H, m, H-11), 1.90 (1H, m, H-16), 1.86 (1H, m, H-5), 1.63 (3H, s, H-21), 1.54 (3H, s, H-20), 1.25 (3H, d, J=7.3 Hz, H-19), 1.18 (3H, d, J=6.9 Hz, H-22). ¹³C-NMR(125 MHz, CDCl₃) δ (ppm): 197.1 (C-1), 180.3 (C-18), 170.0 (CH₃CO), 158.4 (C-3), 137.7 (C-9), 137.6 (C-2), 130.4 (C-13), 128.5 (C-12), 121.0 (C-8), 77.1 (C-15), 69.4 (C-4), 61.0 (C-23), 60.0 (C-24), 45.2 (C-14), 43.3 (C-5), 41.6 (C-6), 39.6 (C-10), 34.9 (C-16), 33.9 (C-17), 27.1 (C-11), 26.5 (C-7), 21.2 (CH₃CO), 16.7 (C-21), 16.3 (C-20), 16.1 (C-19), 13.1 (C-22).

### Drug Toxicity Tests

In the present embodiment, a specific number of cells were cultured in 25T cell culture medium. After 6 hours, the cells attached to the bottom of the medium. Drugs (A. camphorata extracts) of 0, 50, 100, 200, and 400 nM were added when the cells remained in undivided state. The medium containing the drugs were removed at 0, 6, 12 and 24 hours after incubation. Cells were washed with PBS once and replenished with the drug-free fresh medium, then cultured for 10 to 14 days. The cultured cells were fixed with methanol and stained with Giemsa, and then the number of cell colonies were counted (each colony must contain more than 50 cells). The surviving fractions (SF) were calculated as:

$$S {F}_{xnM , thr} = \frac{{PE}_{xnM , thr}}{{PE}_{enM , thr}} ( S F \text{:} surviving fraction , PE \text{:} plating efficiency )$$

Combining with Radiation Treatment

A specific number of cells were cultured in 25T cell culture medium. After the cells attached to the bottom of the medium, the medium was replaced and 200 nM of the fresh cell culture medium was added. Cells were cultured for 24 hours and then irradiated with radiation. The cell culture medium was replaced immediately after irradiation. The cells were cultured for further 10 to 14 days and then stained with Giemsa. The number of cell colonies (each colony must contain more than 50 cells) were counted. The surviving fractions (SF) were calculated as:

$$S {F}_{xnM , DGy} = \frac{{PE}_{xnM , DGy}}{{PE}_{enM , oGy}} ( S F \text{:} surviving fraction , PE \text{:} Plating efficiency )$$

Different cancer stem cells were used to test for cytotoxic concentration of co-administration of radiation therapy with five A. camphorata extracts based on the half maximal inhibitory concentration (IC₅₀) of D1˜D5.

### Cytotoxic Activity Test (MTT Assay)

The principle of cytotoxic activity test (MTT assay) is that succinate dehydrogenase in the mitochondria of a living cell can reduce MTT (3-)4,5-dimethylthiazol-2-yl)-2,5-diphenyltetrazolium bromide), and blue-violet formazan was formed under reaction with cytochrome C. Generally, the amount of formazan generated is proportional to the activity of the mitochondria and the number of living cells. After adding dimethyl sulfoxide (DMSO) to dissolve formazan, the number of living cells can be estimated by the optical density (OD) value.

### Half Maximal Inhibitory Concentration (IC₅₀)

The definition of half maximal inhibitory concentration (IC₅₀) is the concentration for survival rate of 50% under the reaction with drugs or compounds.

### Chemotherapy Drug Standards

Cisplatin: cis-diammineplatinum(II) dichloride (Sigma-Aldrich, USA) Taxol: Paclitaxel (Sigma-Aldrich, USA)

Combining with Chemotherapy Drugs

According to the pretested half maximal inhibitory concentration (IC₅₀) of D1˜D5, different cancer stem cells were used to test the cytotoxic concentration for (1) the co-administration of A. camphorata extracts with the chemotherapy drug, Cisplatin; (2) the co-administration of A. camphorata extracts with the chemotherapy drug, Taxol.

The inhibitory Ability of A. Camphorata Extracts for Tumor Cells

To assess the ability of A. camphorata extracts against lung cancer, brain cancer, head and neck cancer, colorectal cancer, and breast cancer for inhibiting the growth of cancer stem cells, five A. camphorata extracts were tested as follows: A. camphorata concentrate (D1), ethyl acetate extracts of A. camphorata concentrate (D2), lyophilized powder of A. camphorata concentrate (D3), ethyl acetate extracts of lyophilized powder of A. camphorata concentrate (D4), ethyl acetate extracts of A. camphorata mycelium (D5). The aim of this embodiment was to screen for the A. camphorata extracts with anti-cancer effect. MTT (3-(4,5-dimethylthiazol-2-yl)-2,5-diphenyltetrazolium bromide) assay was used to conduct the cytotoxicity test. Since the dead cells do not have succinate dehydrogenase, there is no reaction after adding MTT. Two human fibroblast cells, AF-1 (Adult fibroblast-1) and AF-2 (Adult fibroblast-2), human lung cancer stem cell (Lung CSC), Glioblastomas multiform cancer stem cells (GBM CSC), Head and neck squamous cell carcinoma cancer stem cells (HNSCC CSC), colorectal cancer stem cells (CRC CSC), breast cancer stem cells (Breast CSC) were used as cell experimental models.

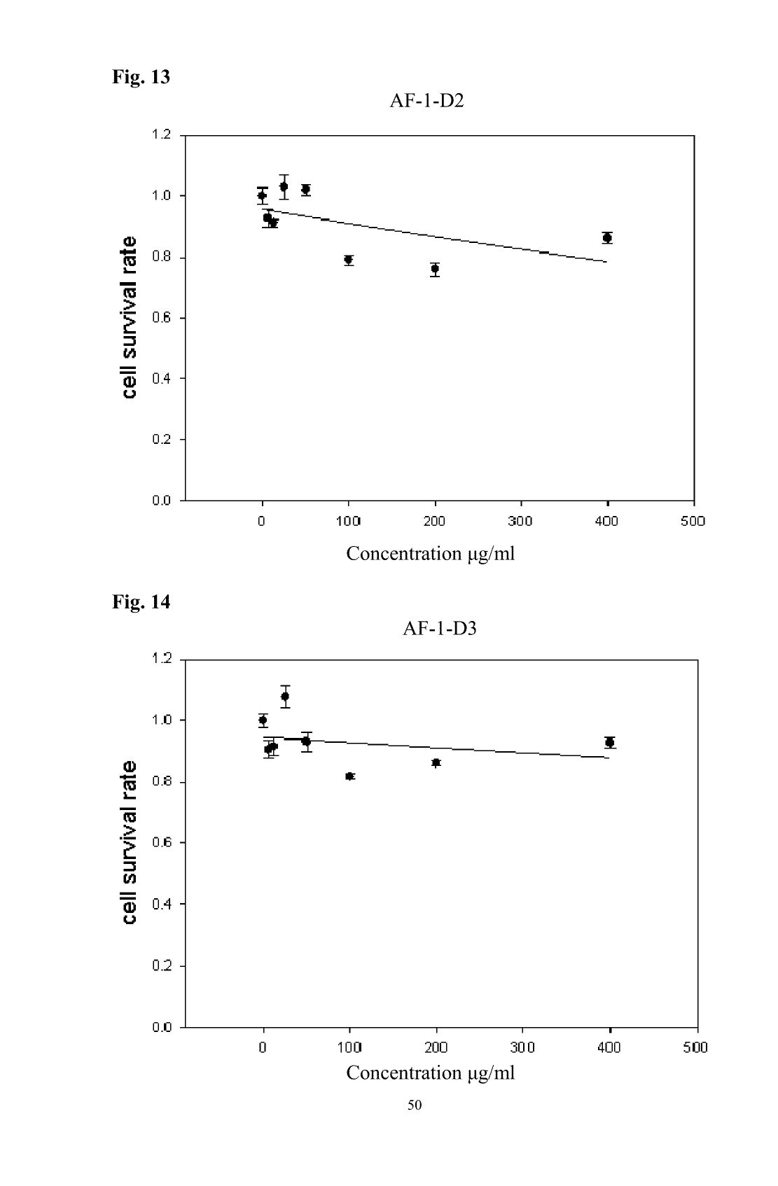

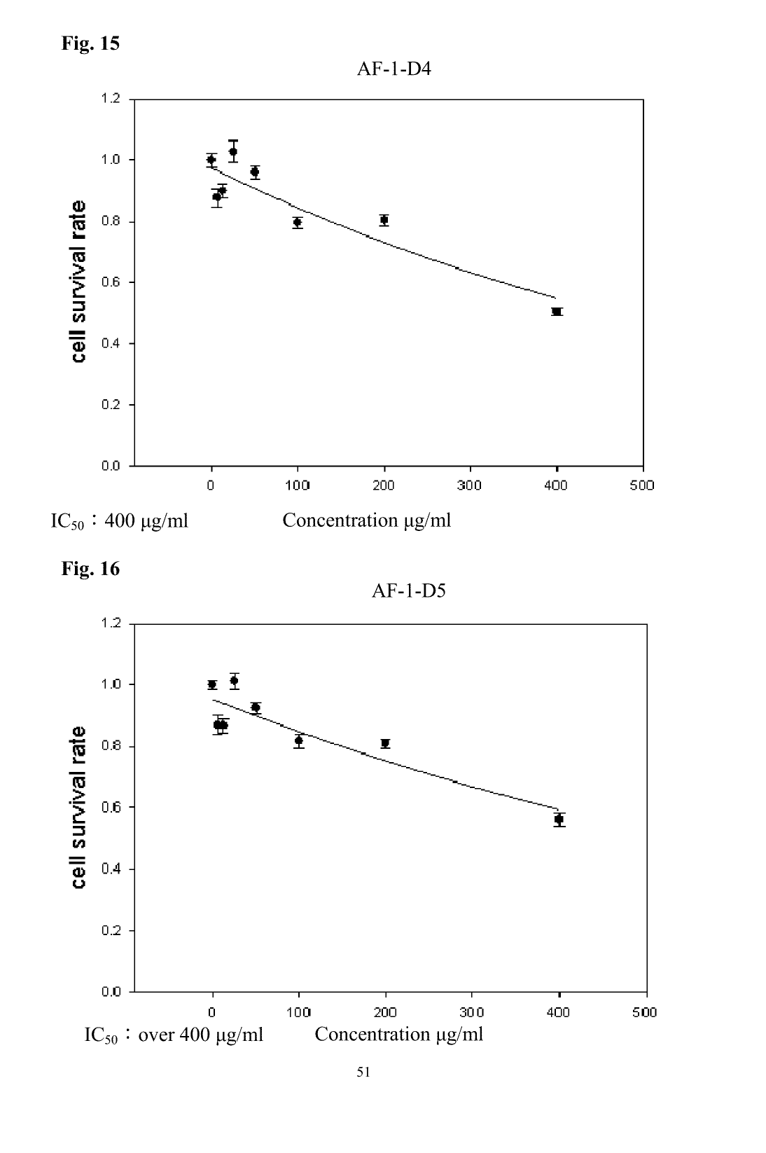

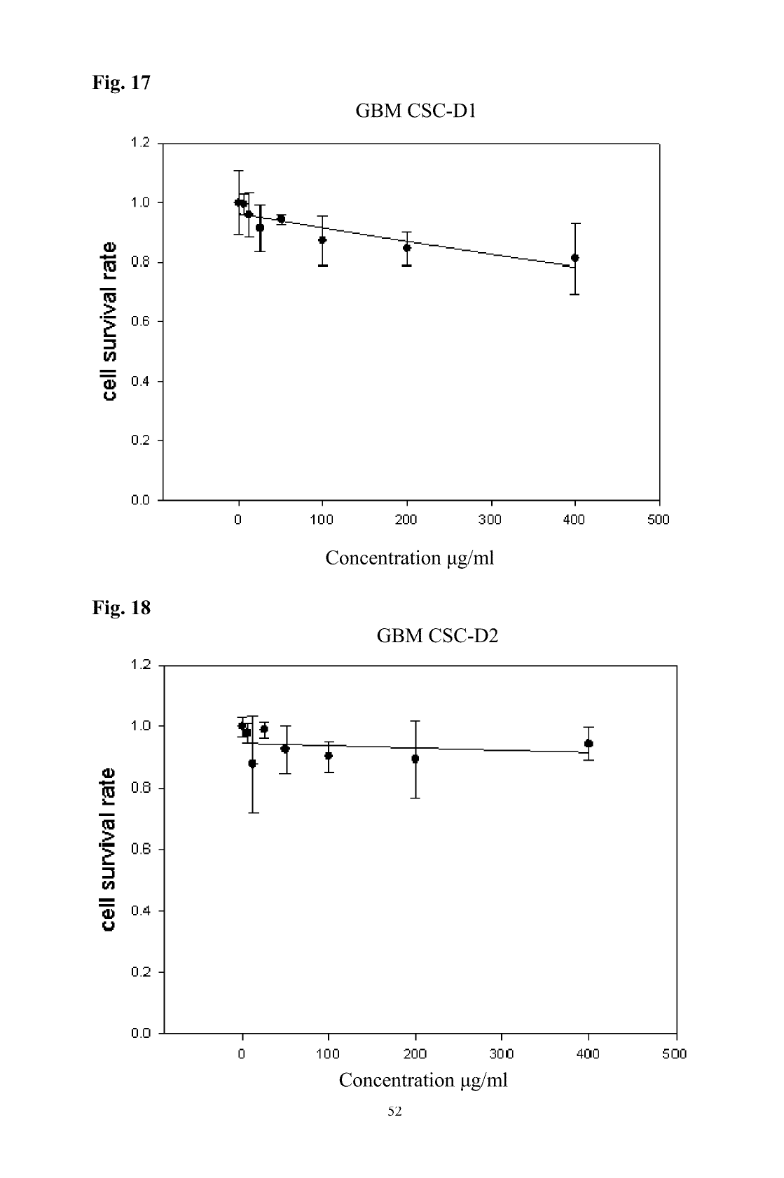

In the initial screening results of A. camphorata extracts, ethyl acetate extracts of lyophilized powder of A. camphorata concentrate (D4) and ethyl acetate extracts of A. camphorata mycelium (D5) had the best growth inhibitory effects for human lung cancer stem cell (Lung CSC), as shown in FIGS. 10, 11; ethyl acetate extracts of lyophilized powder of A. camphorata concentrate (D4) and ethyl acetate extracts of A. camphorata mycelium had the best growth inhibitory effects for human fibroblast cells AF-1 (Adult fibroblast-1), as shown in FIGS. 15, 16. In drug screening results, half maximal inhibitory concentration (IC₅₀) was shown in Table 1 that ethyl acetate extracts of lyophilized powder of A. camphorata concentrate (D4) and ethyl acetate extracts of A. camphorata mycelium (D5) had better growth inhibitory effects.

TABLE 1
The half maximal inhibitory concentration (IC₅₀) of A. camphorata
extracts for human fibroblast cells AF-1 (Adult fibroblast-1), AF-2
(Adult fibroblast-2), lung cancer stem cells (Lung CSC),
Glioblastomas multiform cancer stem cells (GBM CSC), Head and neck
squamous cell carcinoma cancer stem cells (HNSCC CSC), colorectal
cancer stem cells (CRC CSC), breast cancer stem cells (Breast CSC),
Hepatoma cancer stem cells (Hepatoma CSC), Leukemia cancer stem
cells (Leukemia CSC) and Gastric stem cells (Gastric CSC).
IC₅₀

| | D1 | D2 | D3 | D4 | D5 |
|---|---|---|---|---|---|
| Adult fibroblast(AF-1) | x | x | x | 400 | Over 400 |
| | | | | μg/ml | μg/ml |
| Adult fibroblast (AF-2) | x | x | x | 164.6 | 224.3 |
| | | | | μg/ml | μg/ml |
| Lung cancer stem cells (Lung | x | x | x | 79.7 | 86.9 |
| CSC) | | | | μg/ml | μg/ml |
| Hepatoma cancer stem cells | x | x | x | 167.8 | 268.7 |
| (Hepatoma CSC) | | | | μg/ml | μg/ml |
| Colorectal cancer stem cells | x | x | x | 173.4 | 191.2 |
| (CRC CSC) | | | | μg/ml | μg/ml |
| Breast cancer stem cells (Breast | x | x | x | 55.8 | 168.6 |
| CSC) | | | | μg/ml | μg/ml |
| Leukemia cancer stem cells | x | x | x | 123.7 | 258.9 |
| (Leukemia CSC) | | | | μg/ml | μg/ml |
| Gastric cancer stem cells(Gastric | x | x | x | 119.3 | 210.5 |
| CSC) | | | | μg/ml | μg/ml |
| Glioblastomas multiform cancer | x | x | x | 85.5 | x |
| stem cells (GBM CSC-1) | | | | μg/ml | |
| Glioblastomas multiform stem | x | x | x | 132.6 | 287.8 |
| cells (GBM CSC-2) | | | | μg/ml | μg/ml |
| Head and neck squamous cell | x | x | x | 50.9 | 97.7 |
| carcinoma cancer stem cells | | | | μg/ml | μg/ml |
| (HNSCC CSC) | | | | | |

“x”: The maximum drug concentration 400 μg/ml still can not effectively inhibit more than 50% of the cells

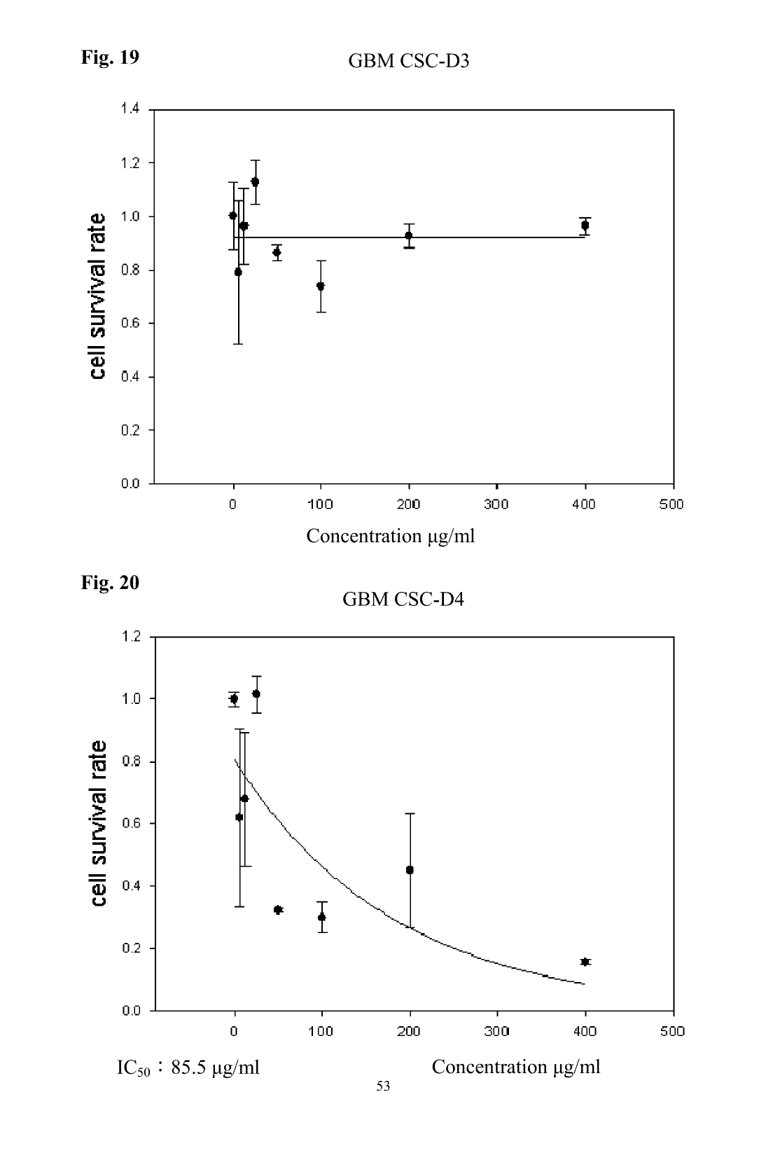

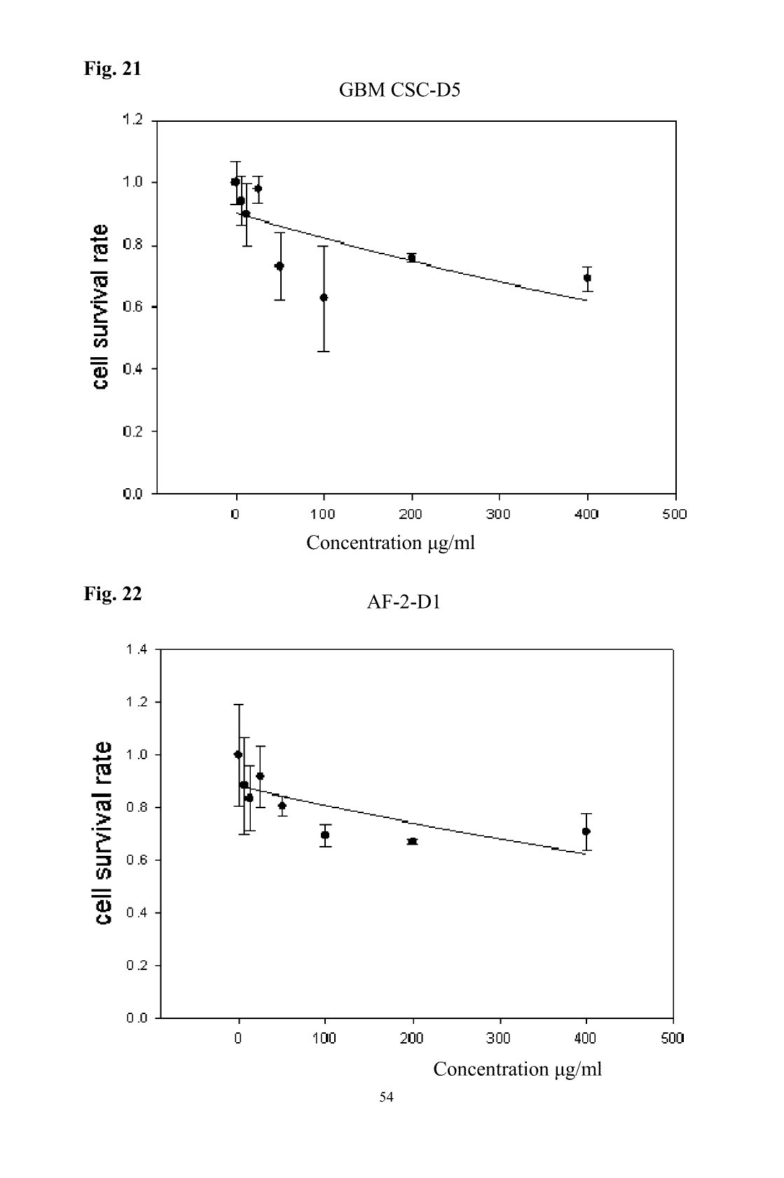

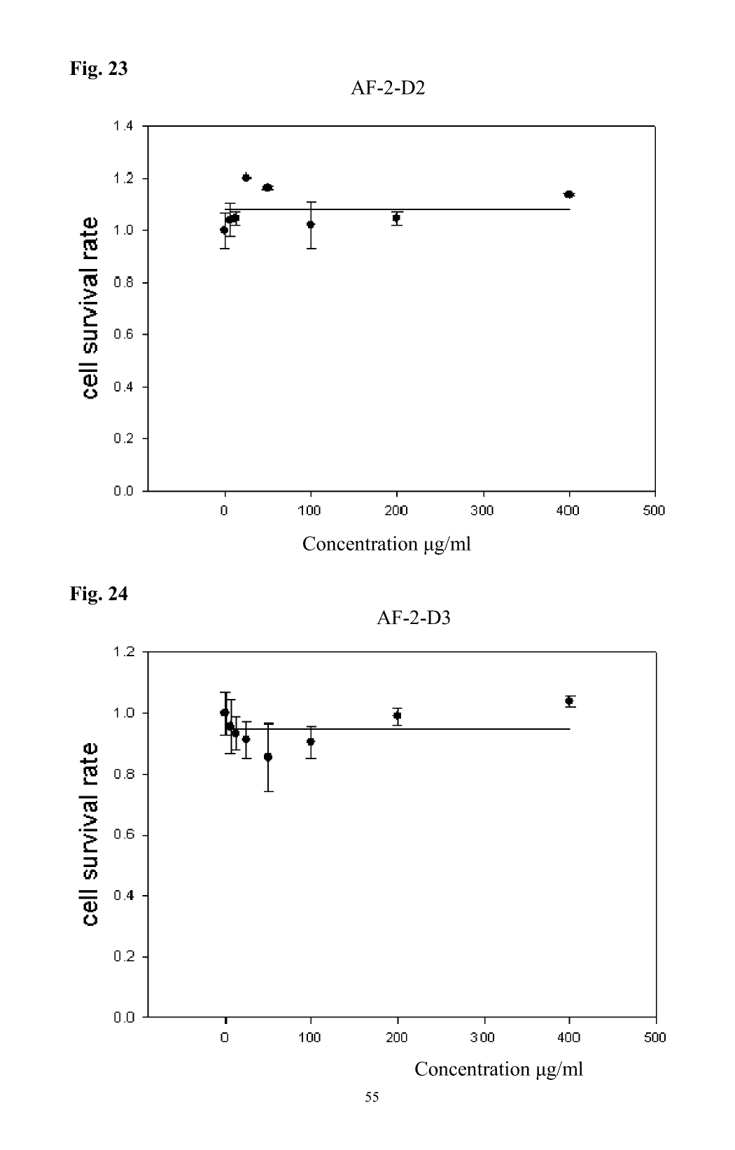

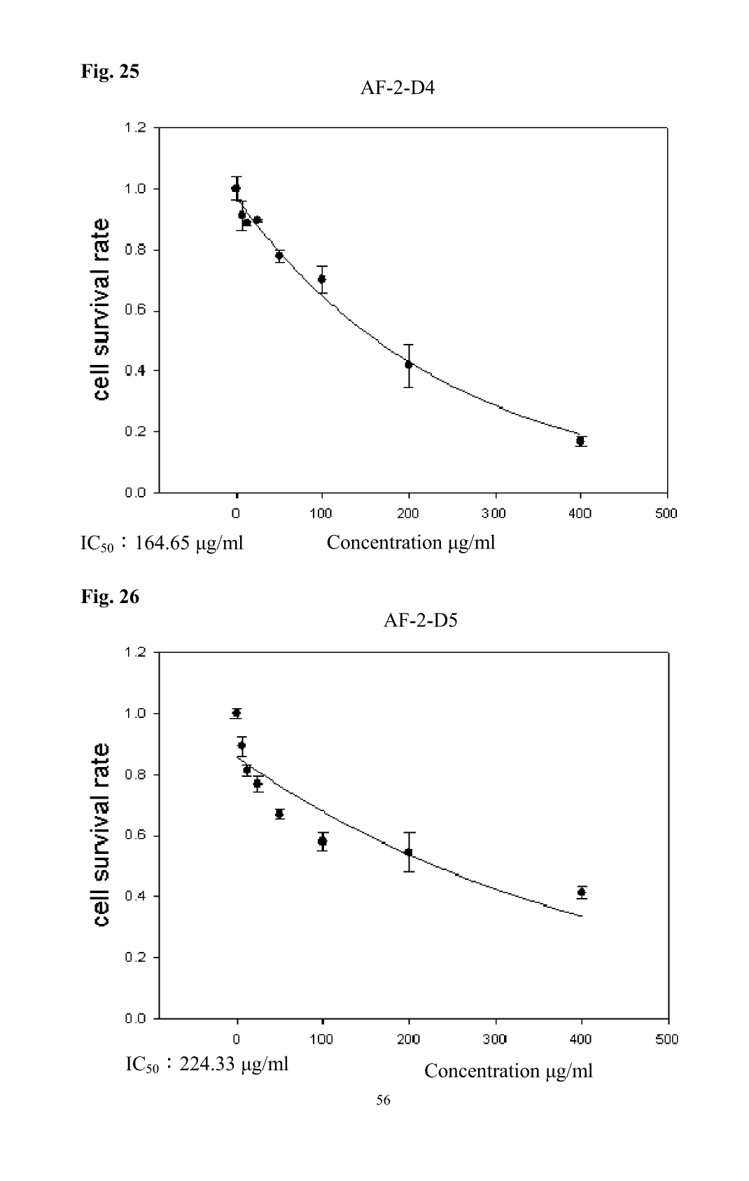

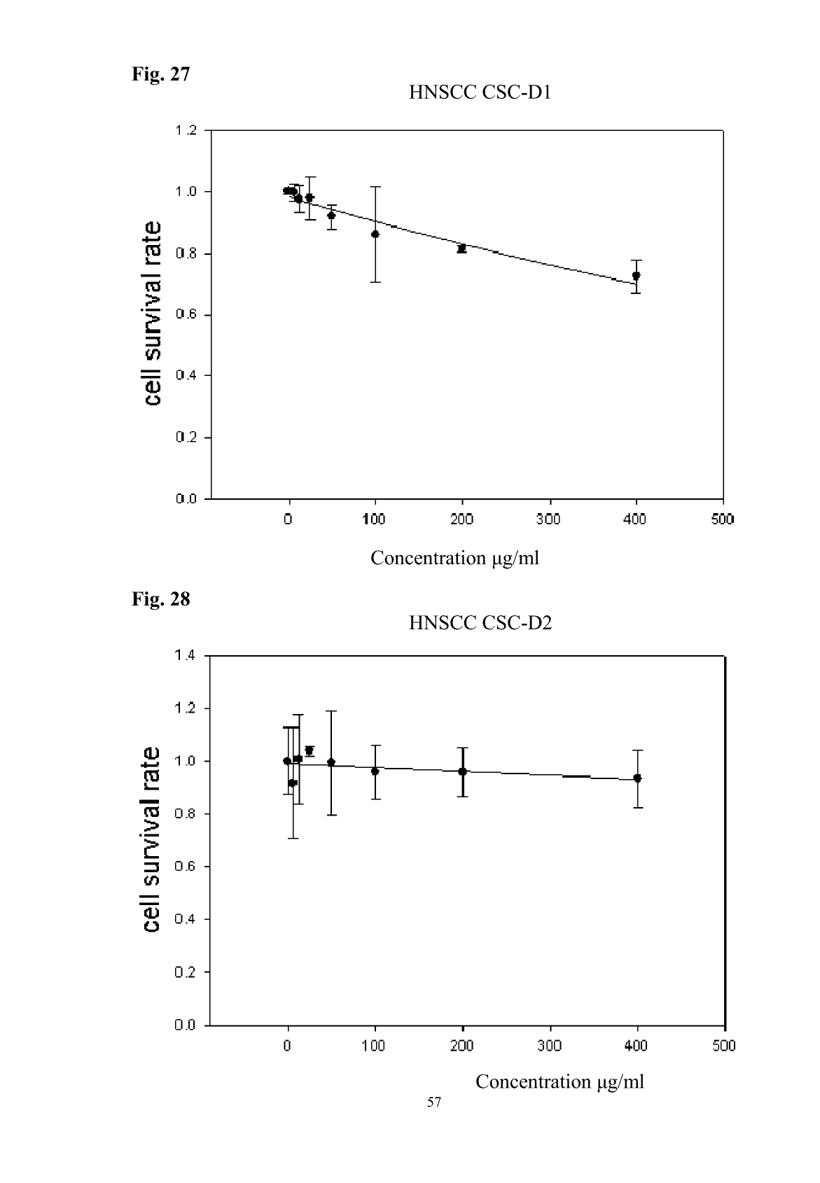

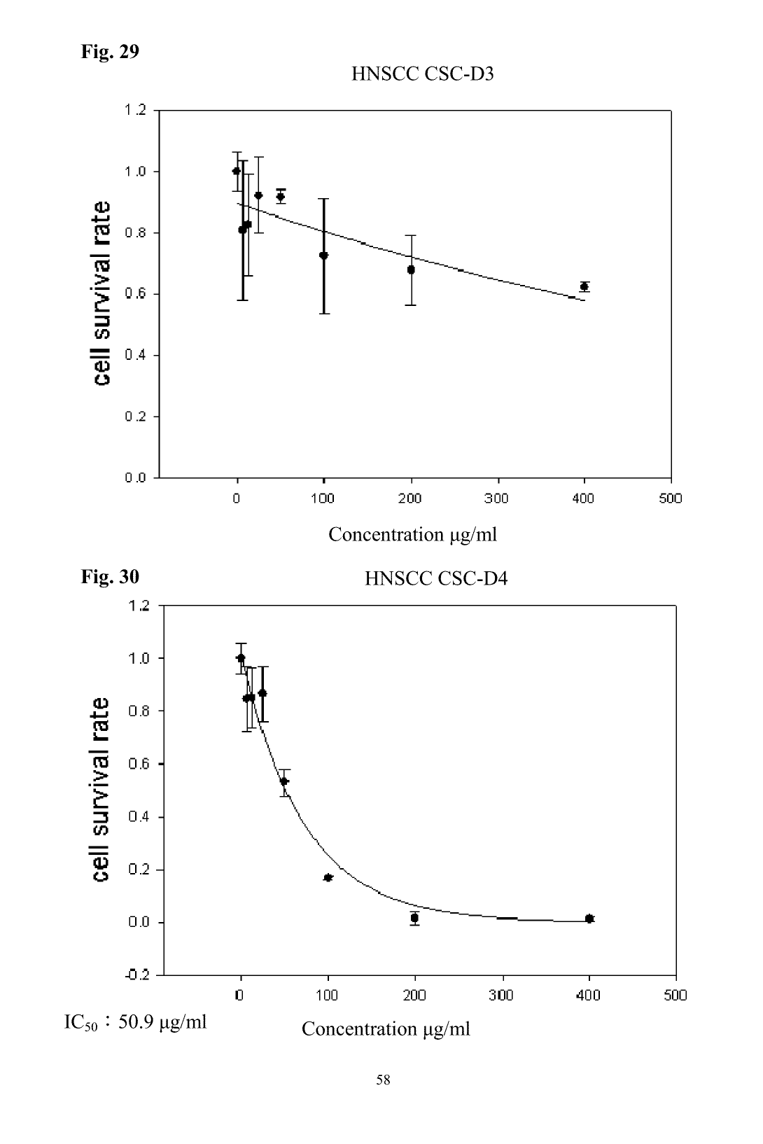

For glioblastoma multiform cancer stem cells, ethyl acetate extracts of lyophilized powder of A. camphorata concentrate (D4) had the best growth inhibitory effects, as shown in FIG. 20; for Adult fibroblast-2 (AF-1), ethyl acetate extracts of lyophilized powder of A. camphorata concentrate (D4) and ethyl acetate extracts of A. camphorata mycelium (D5) had the best growth inhibitory effects, as shown in FIGS. 25, 26.

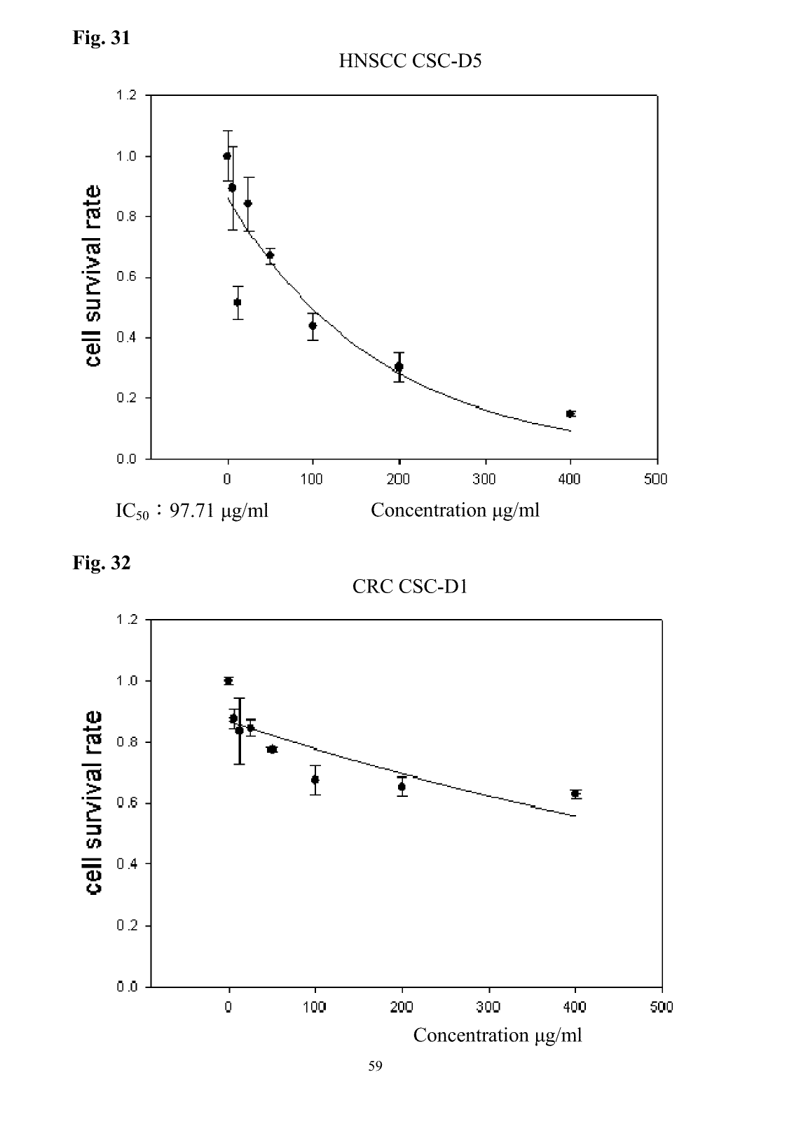

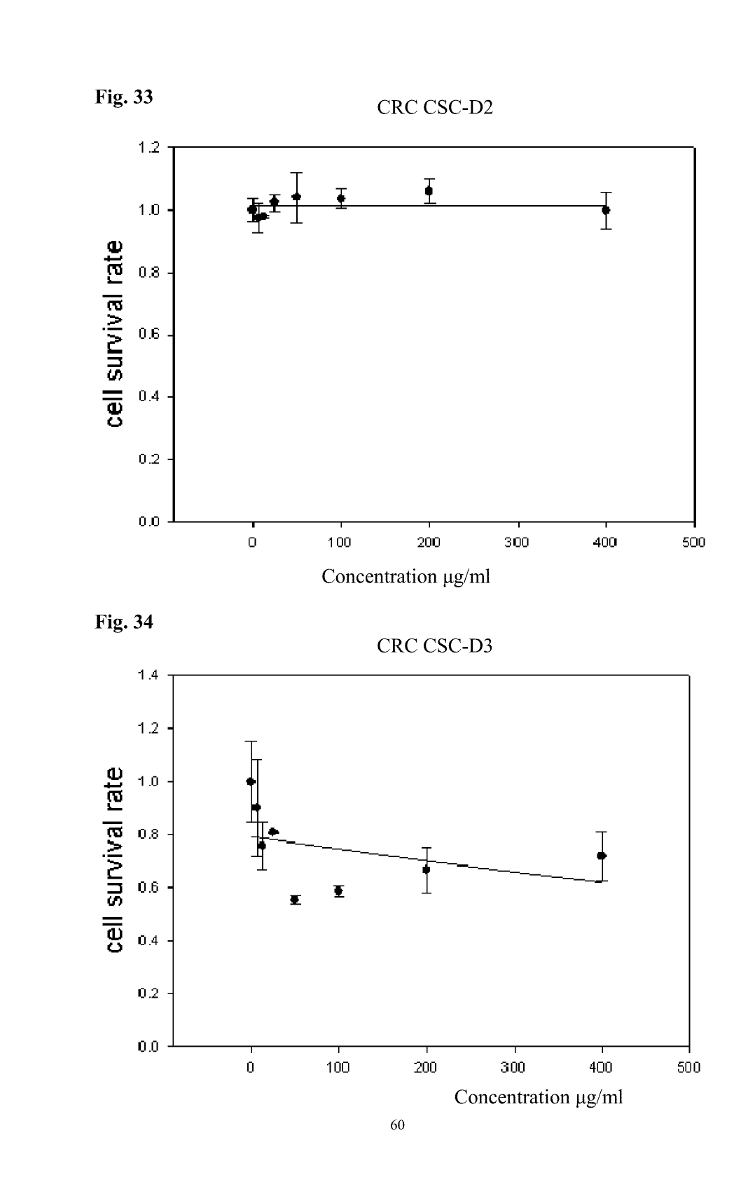

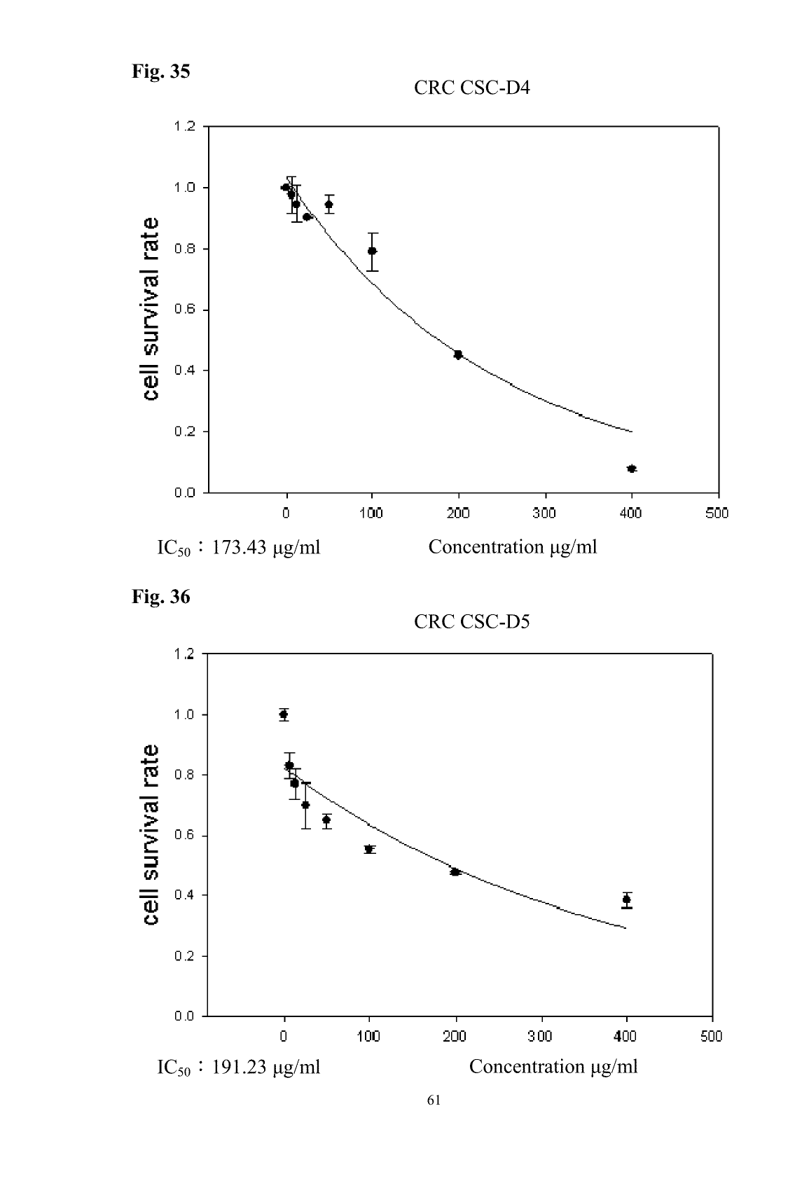

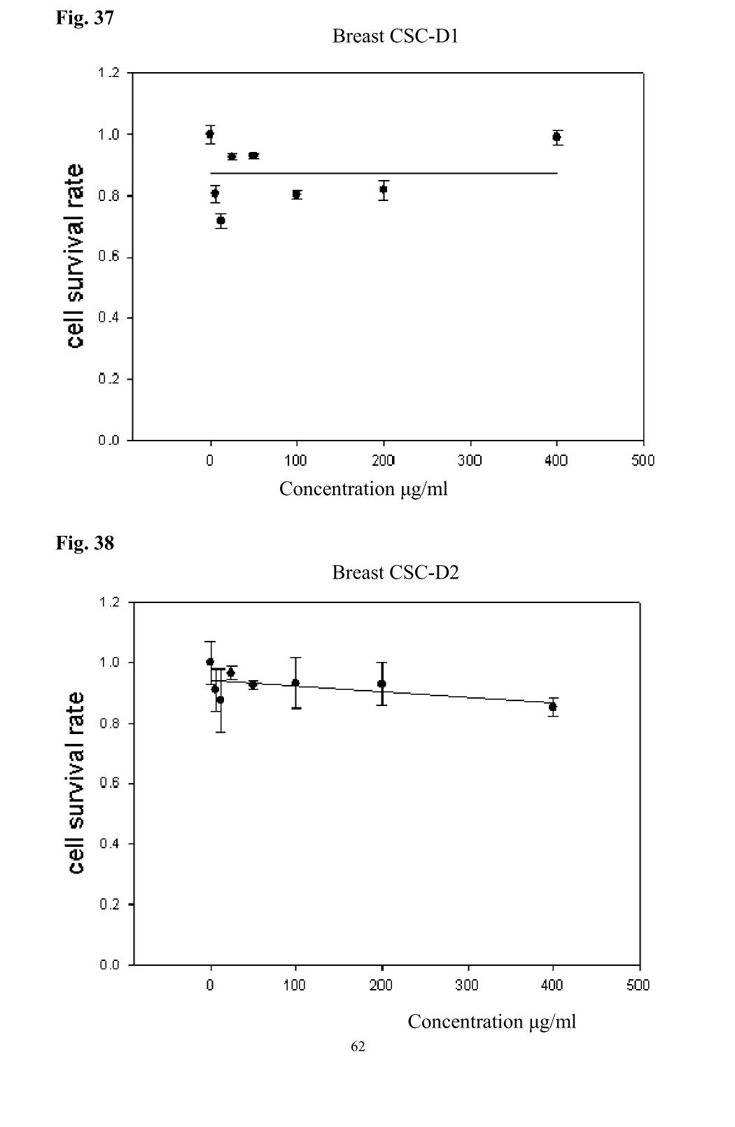

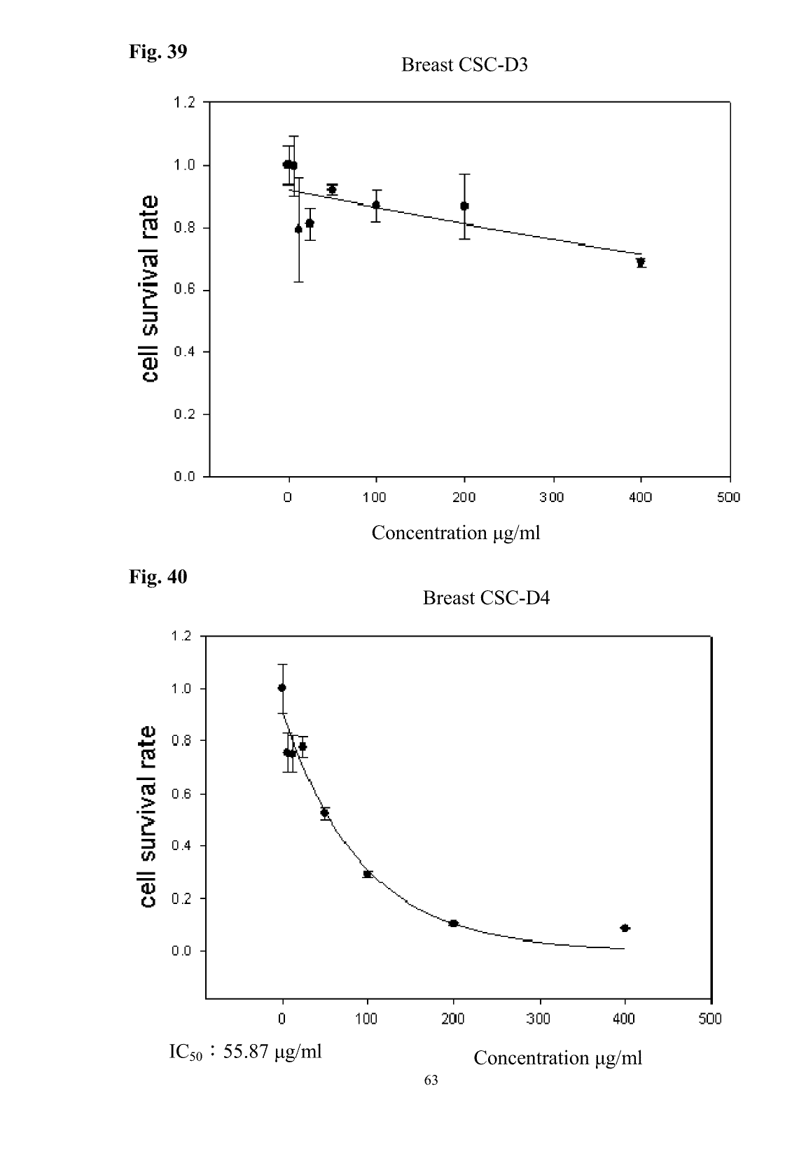

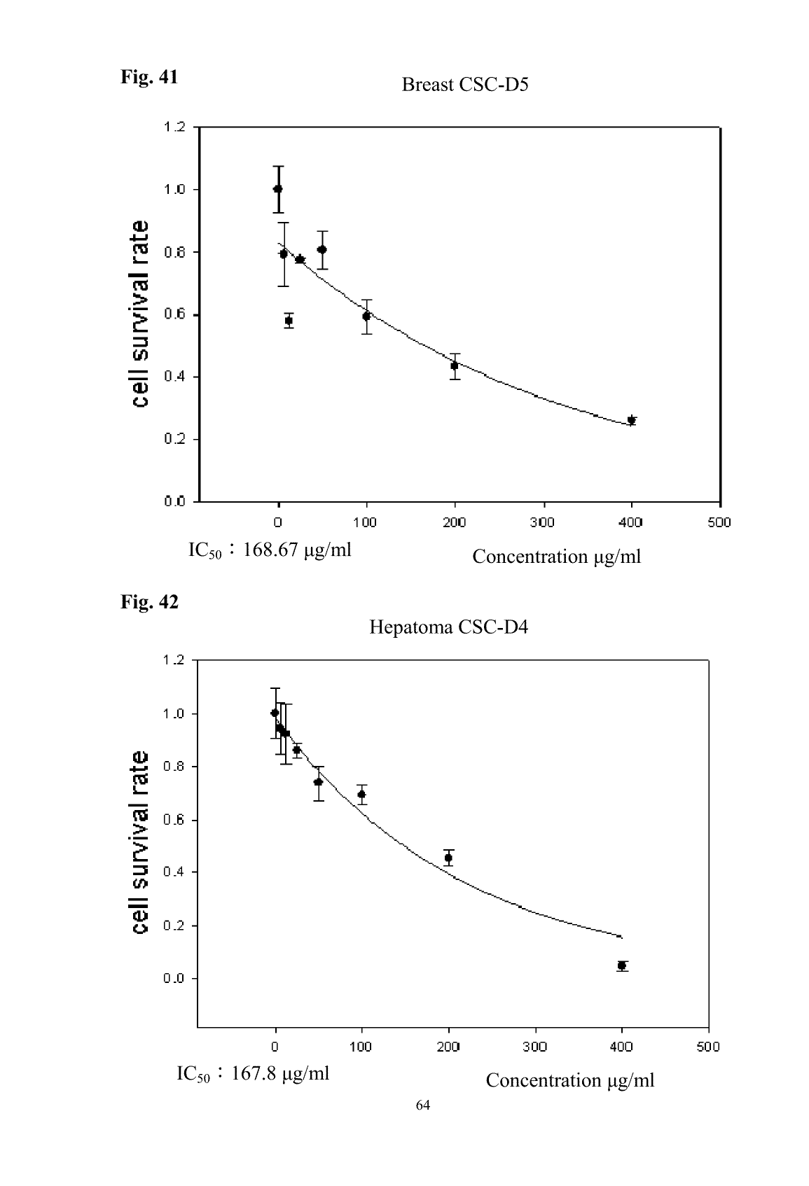

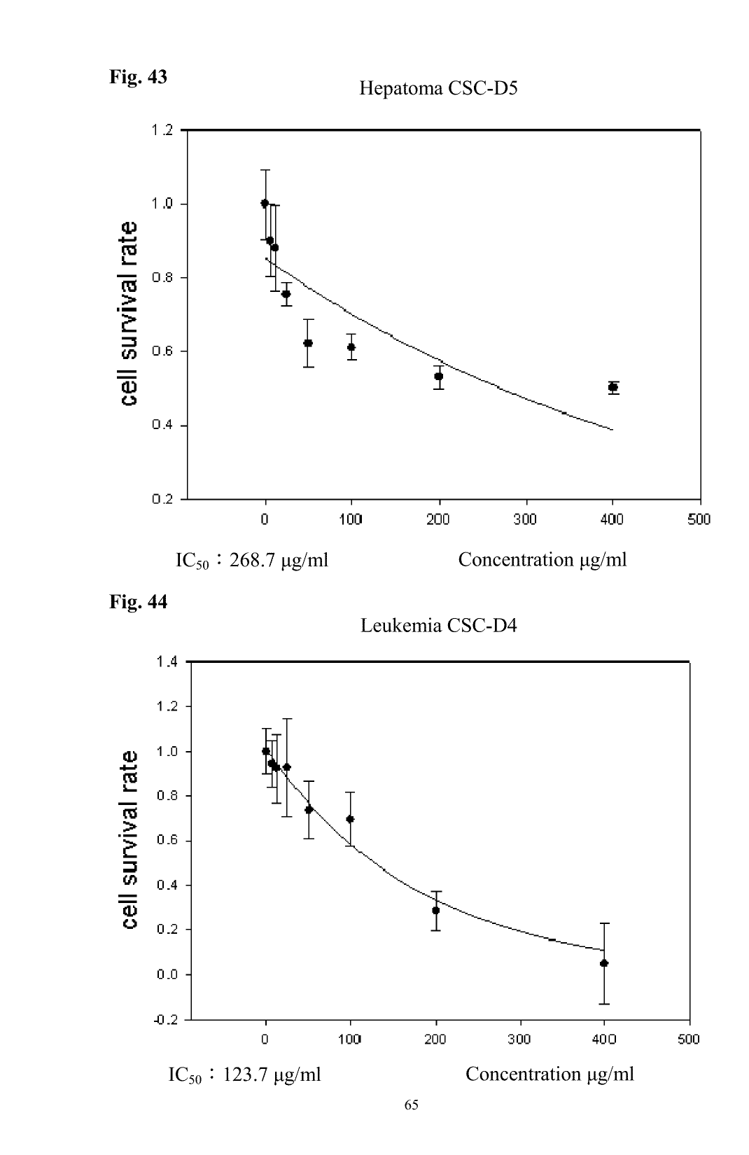

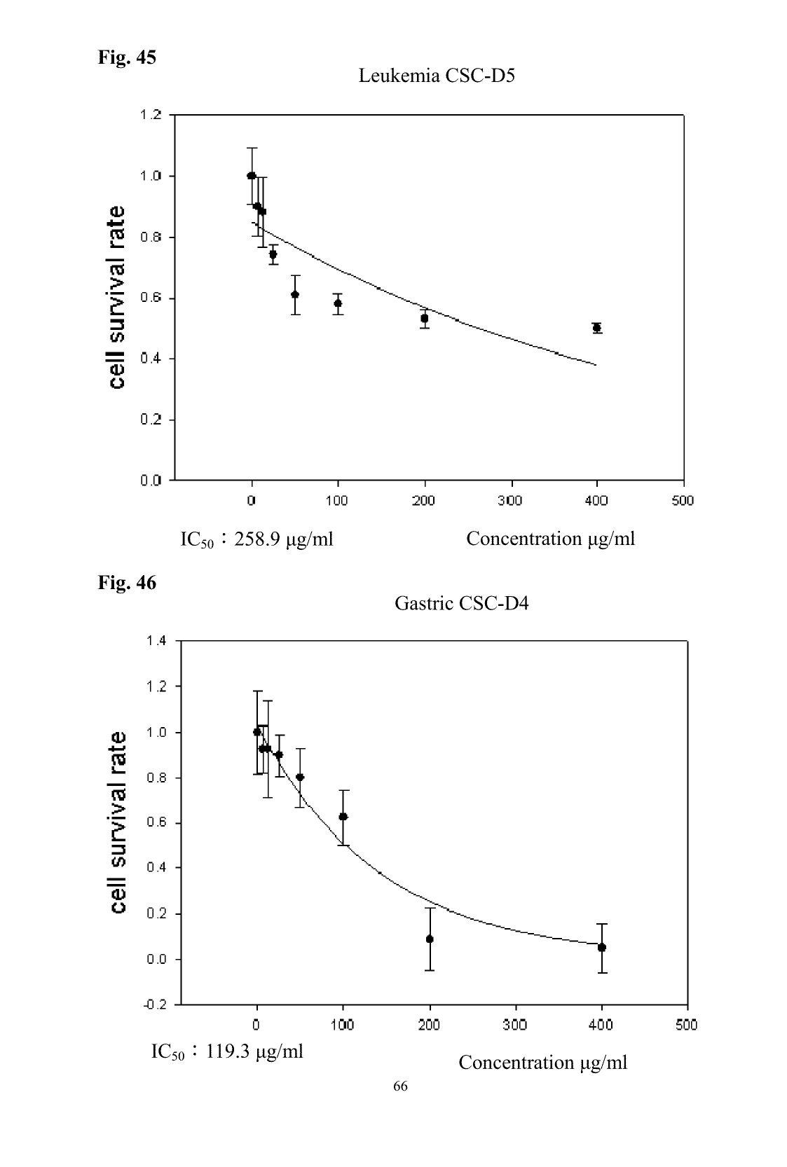

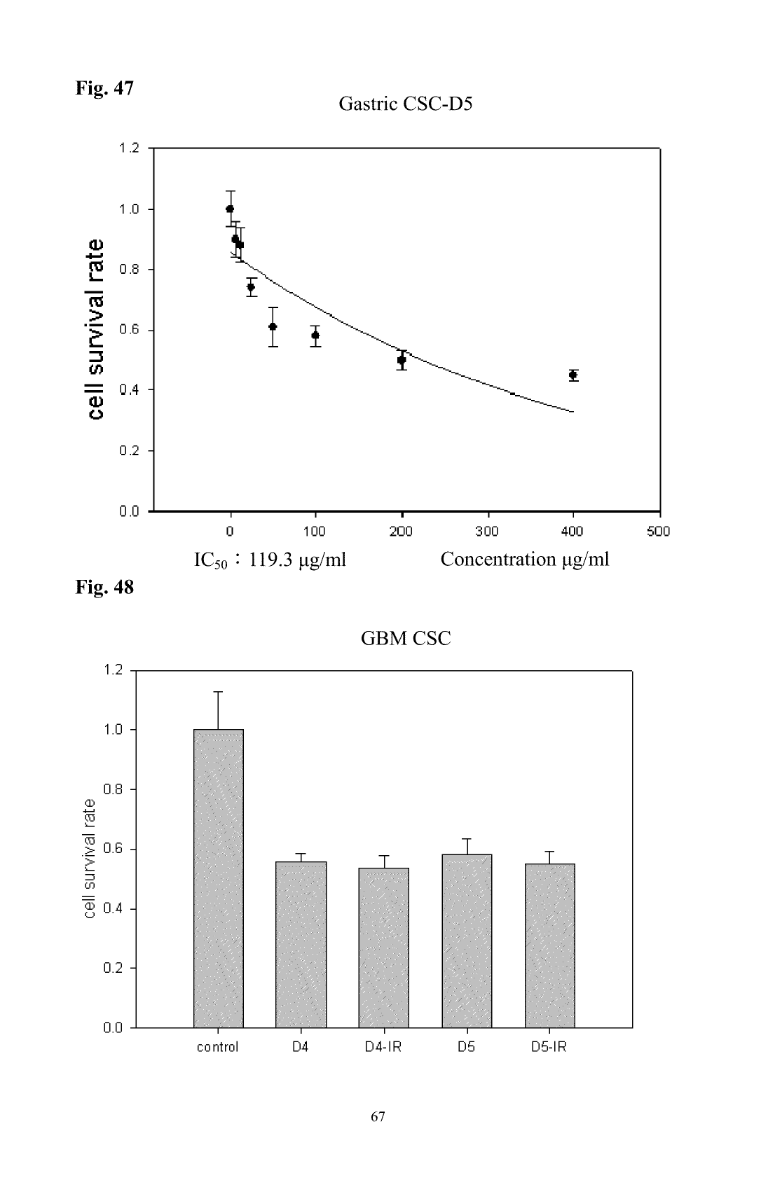

For head and neck squamous cell carcinoma cancer stem cells (HNSCC CSC), ethyl acetate extracts of lyophilized powder of A. camphorata concentrate (D4) and ethyl acetate extracts of A. camphorata mycelium (D5) had the best growth inhibitory effects, as shown in FIGS. 30, 31. For colorectal cancer stem cells (CRC CSC), ethyl acetate extracts of lyophilized powder of A. camphorata concentrate (D4) and ethyl acetate extracts of A. camphorata mycelium (D5) had the best inhibitory growth effects, as shown in FIGS. 35, 36. For Breast cancer stem cells (Breast CSC), ethyl acetate extracts of lyophilized powder of A. camphorata concentrate (D4) and ethyl acetate extracts of A. camphorata mycelium (D5) had the best growth inhibitory effects, as shown in FIGS. 40, 41.

In summary, with respect to the half maximal inhibitory concentration (IC₅₀) of the five A. camphorata extracts (D1-D2-D3-D4-D5) for human fibroblast cells AF-1 (Adult fibroblast-1), AF-2 (Adult fibroblast-2), lung cancer stem cells (Lung CSC), Glioblastomas multiform cancer stem cells (GBM CSC), Head and neck squamous cell carcinoma cancer stem cells (HNSCC CSC), colorectal cancer stem cells (CRC CSC), breast cancer stem cells (Breast CSC), ethyl acetate extracts of lyophilized powder of A. camphorata concentrate (D4) and ethyl acetate extracts of A. camphorata mycelium (D5) had the better growth inhibitory effects, as shown in Table 1.

In Table 1, the inhibitory effects of the growth of lung cancer stem cells (Lung CSC), Glioblastomas multiform cancer stem cells (GBM CSC), Head and neck squamous cell carcinoma cancer stem cells (HNSCC CSC), breast cancer stem cells (Breast CSC) were sensitive to ethyl acetate extracts of lyophilized powder of A. camphorata concentrate (D4) and ethyl acetate extracts of A. camphorata mycelium (D5) which had significant growth inhibitory effects on these four kinds of cancer stem cells. However, in comparison to the cancer stem cells, ethyl acetate extracts of lyophilized powder of A. camphorata concentrate (D4) and ethyl acetate extracts of A. camphorata mycelium (D5) had no significant growth inhibitory effects on normal human fibroblast cells AF-1 (Adult fibroblast-1) and AF-2 (Adult fibroblast-2).

According to the results of the present embodiment, ethyl acetate extracts of lyophilized powder of A. camphorata concentrate (D4) and ethyl acetate extracts of A. camphorata mycelium (D5) contained the ingredients that have the potential to be developed into drugs for cancer stem cells. At the same time, the examination of the cell activity tests and IC₅₀ tests of A. camphorata extracts for normal lung fibroblast were conducted. It is worth noting that the half maximal inhibitory concentration (IC₅₀) of ethyl acetate extracts of lyophilized powder of A. camphorata concentrate (D4) and ethyl acetate extracts of A. camphorata mycelium (D5) for normal lung fibroblast were higher than that for cancer stem cells. These results showed that normal lung fibroblast had higher tolerance for A. camphorata extracts (D4 and D5) (Table 1).

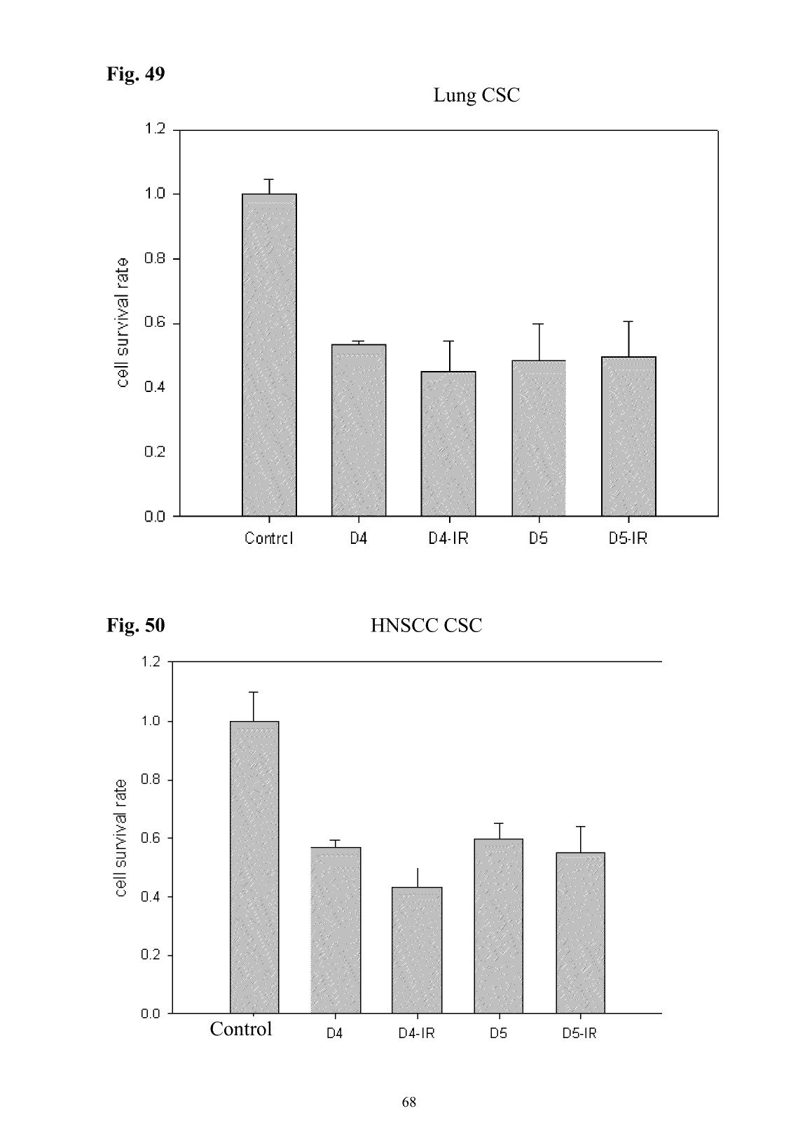

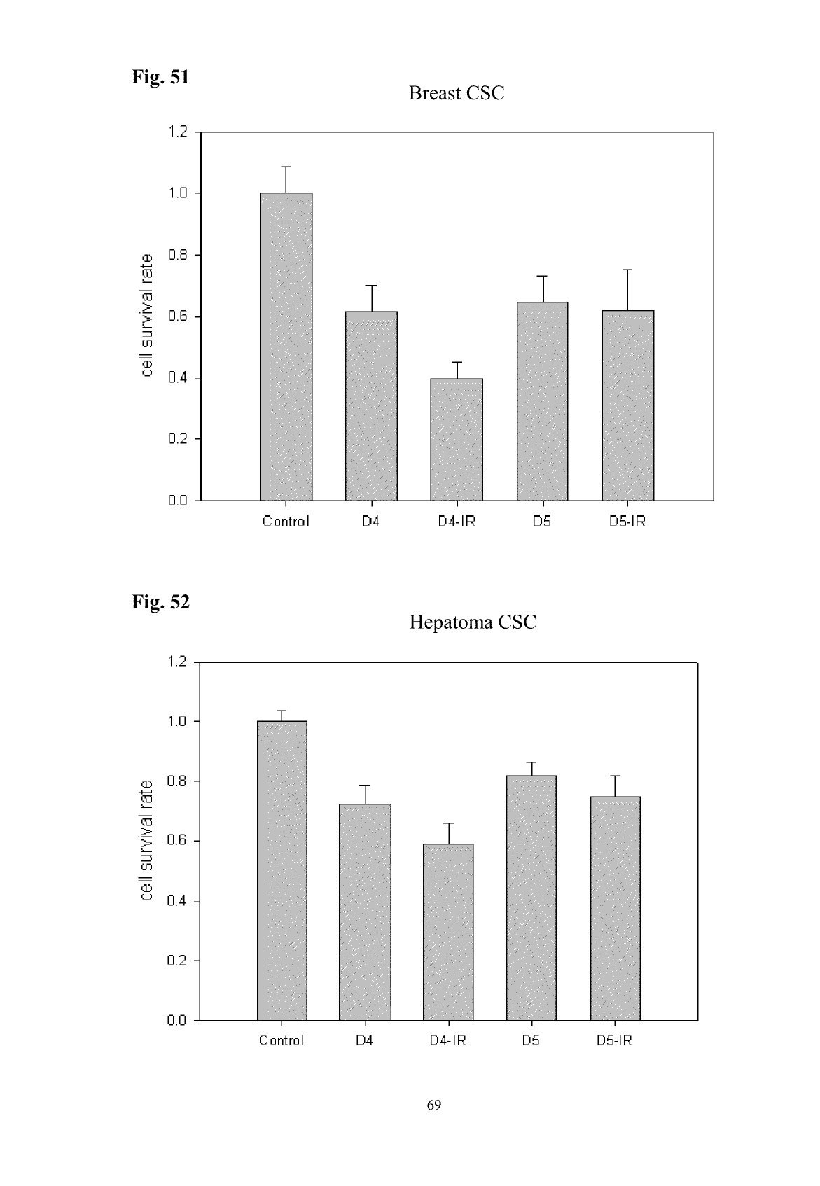

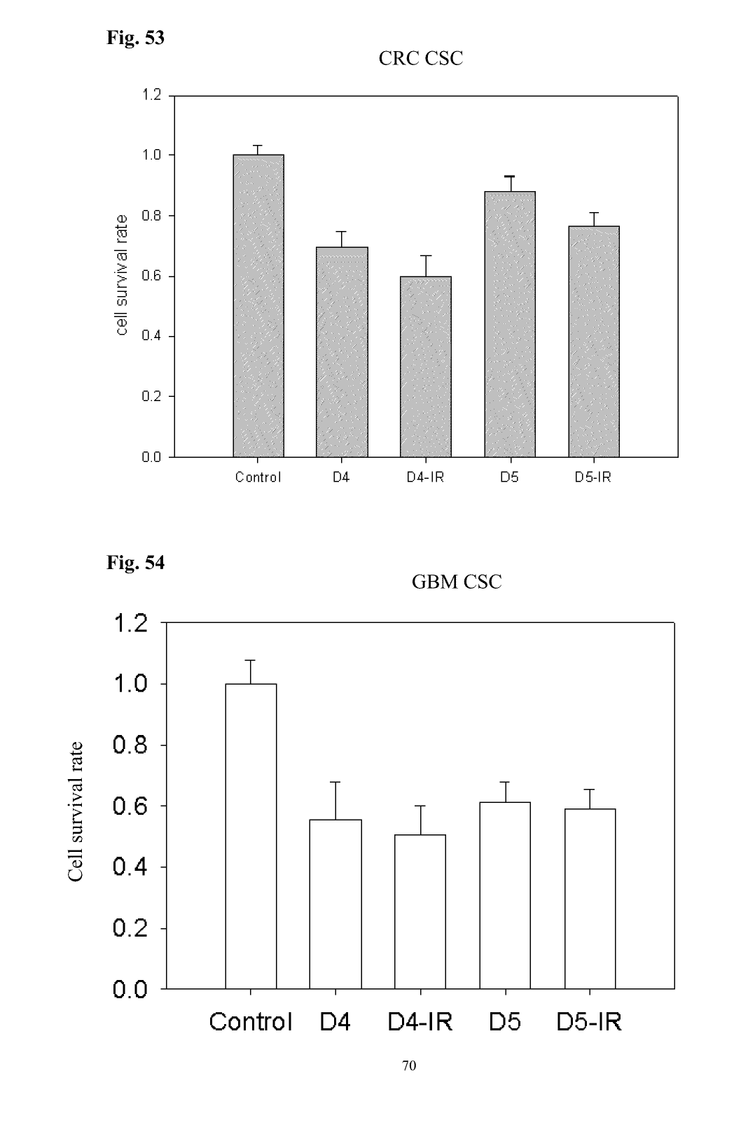

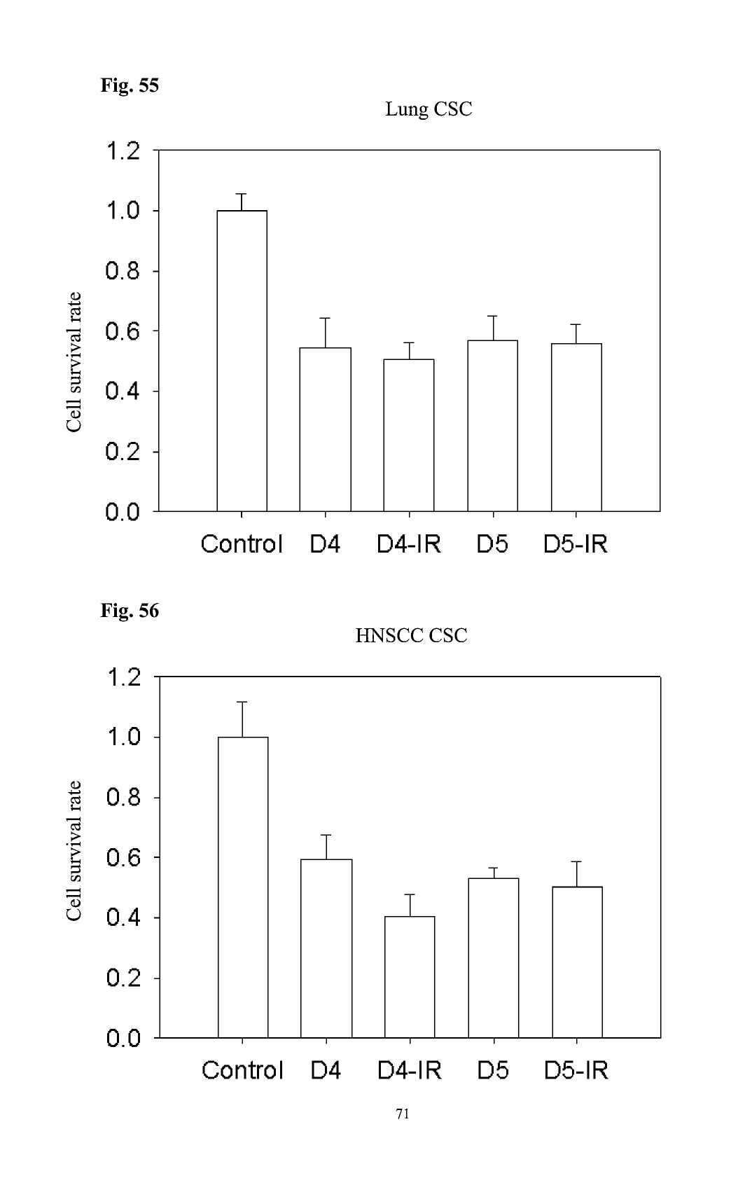

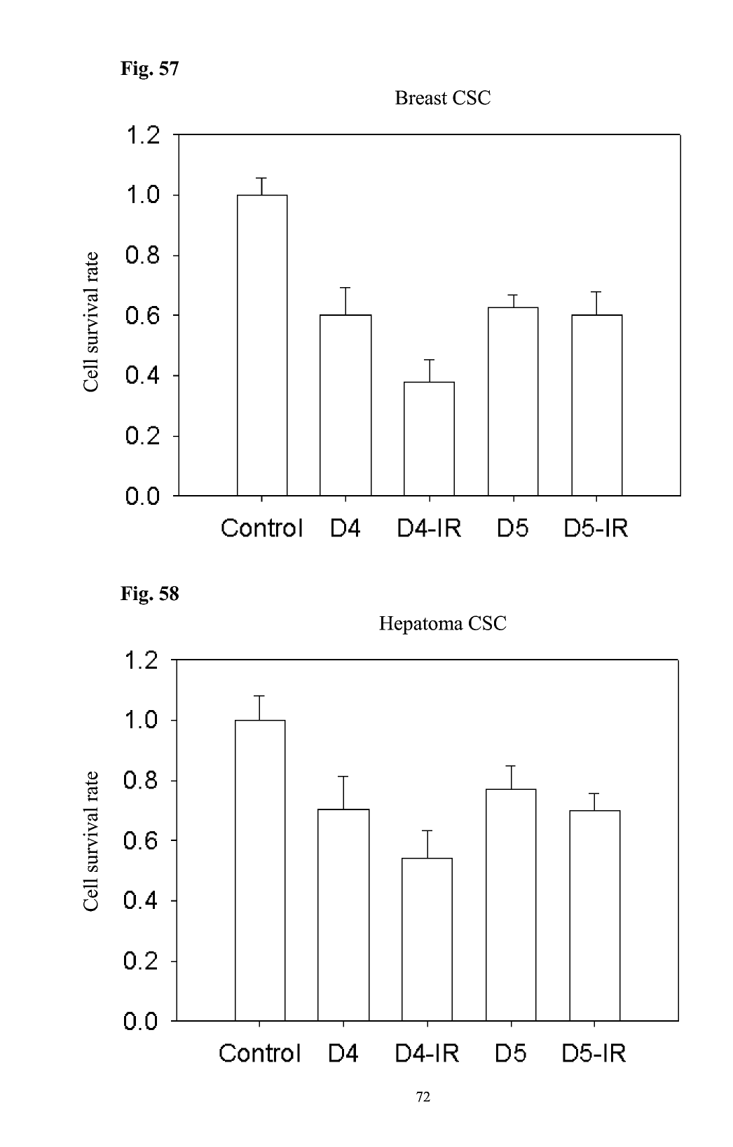

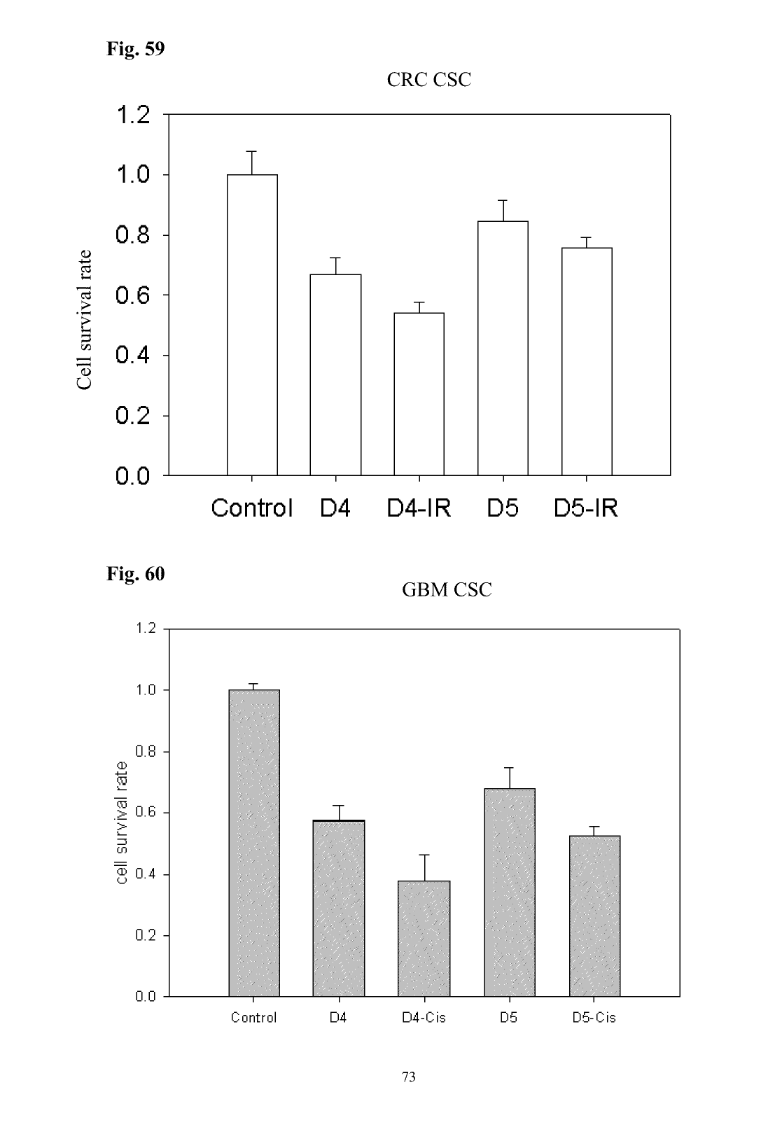

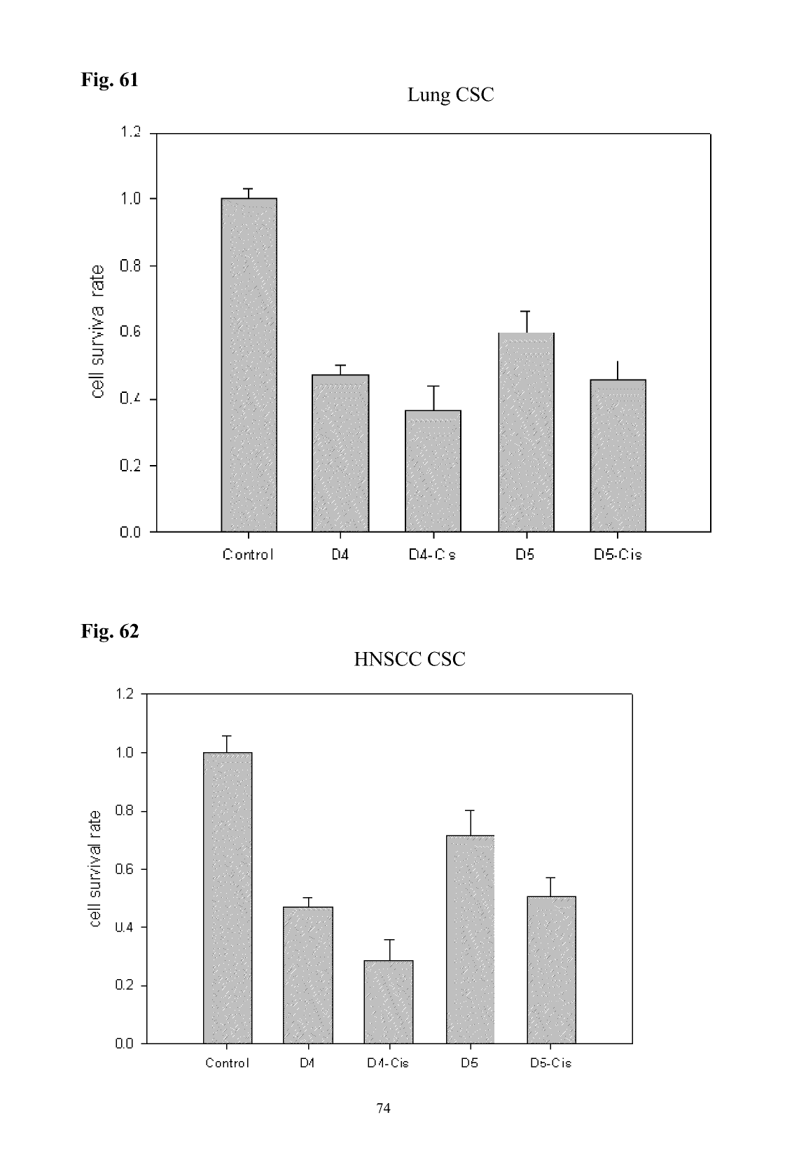

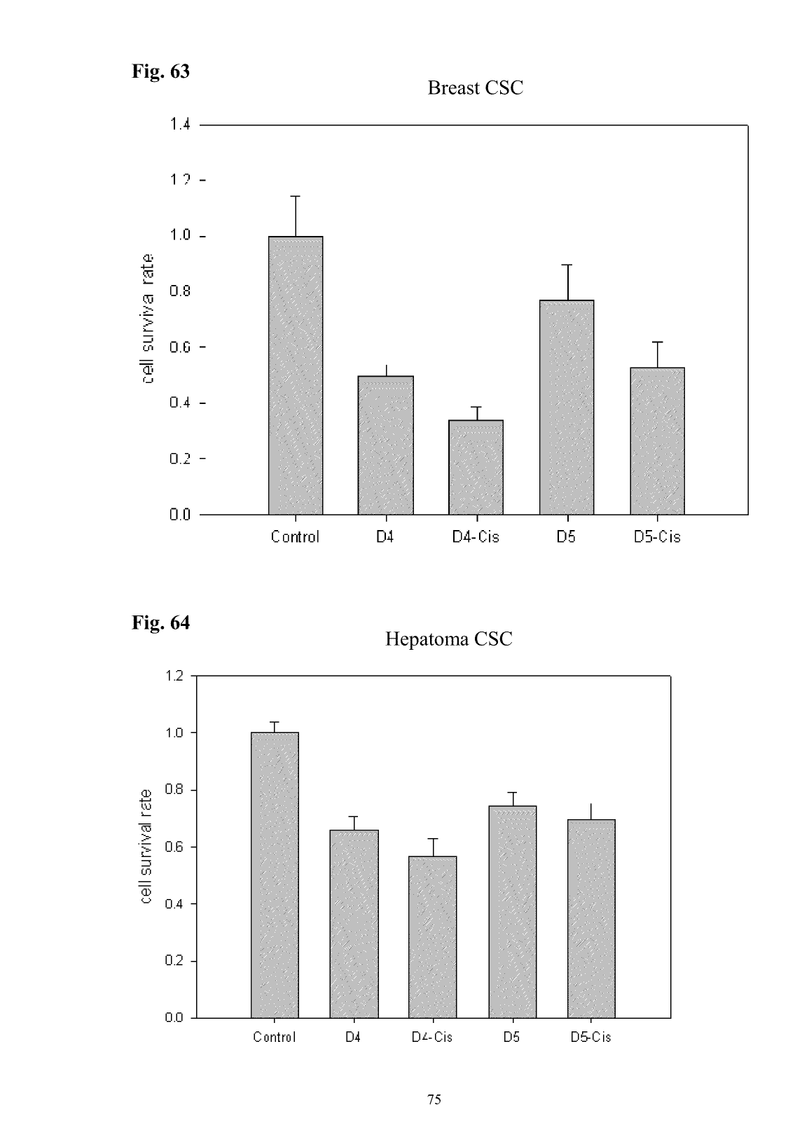

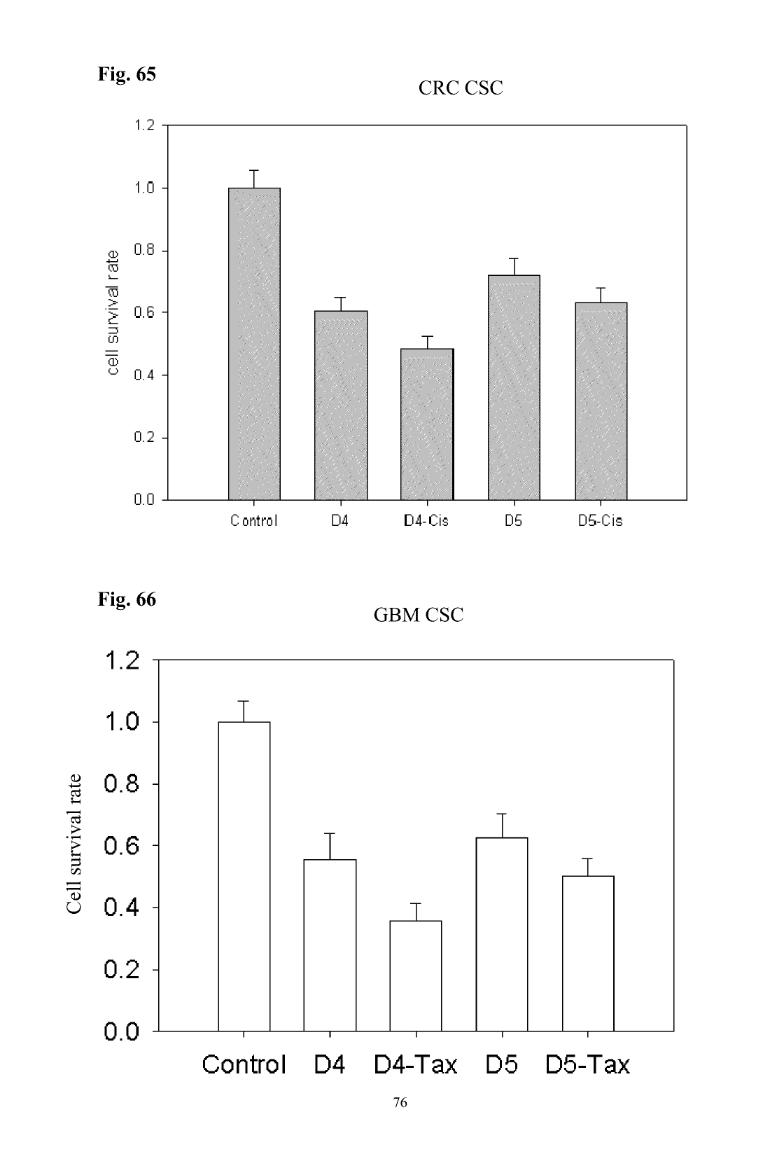

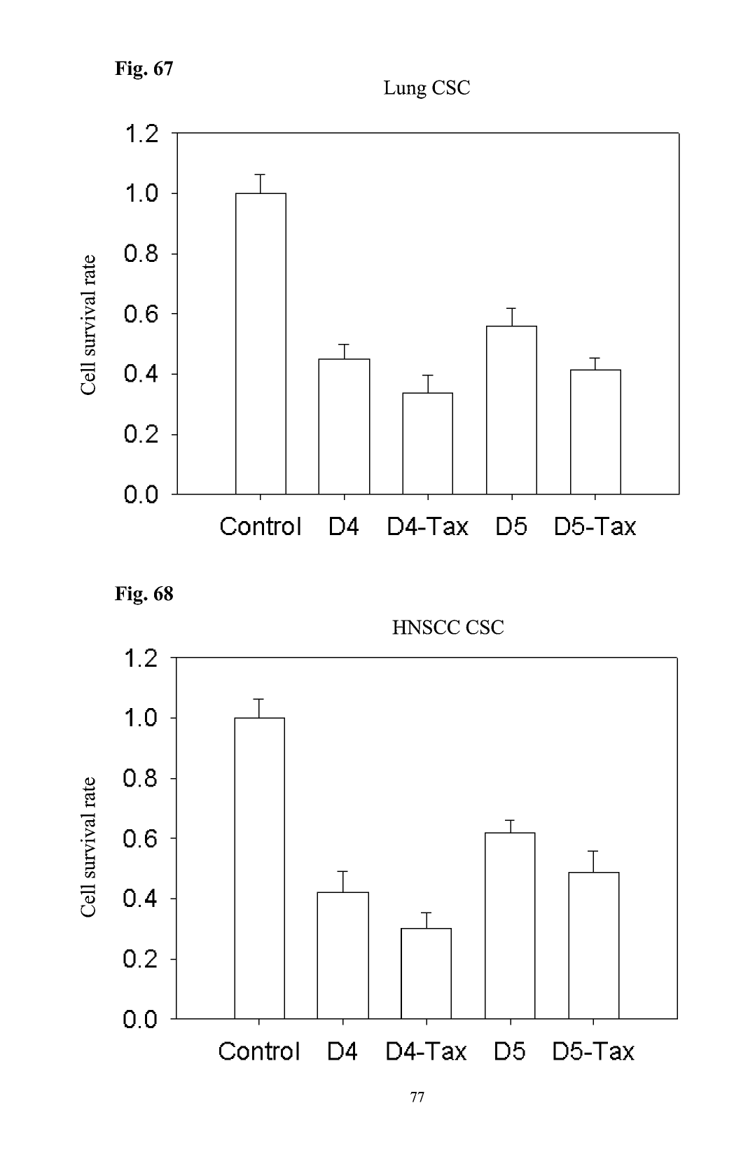

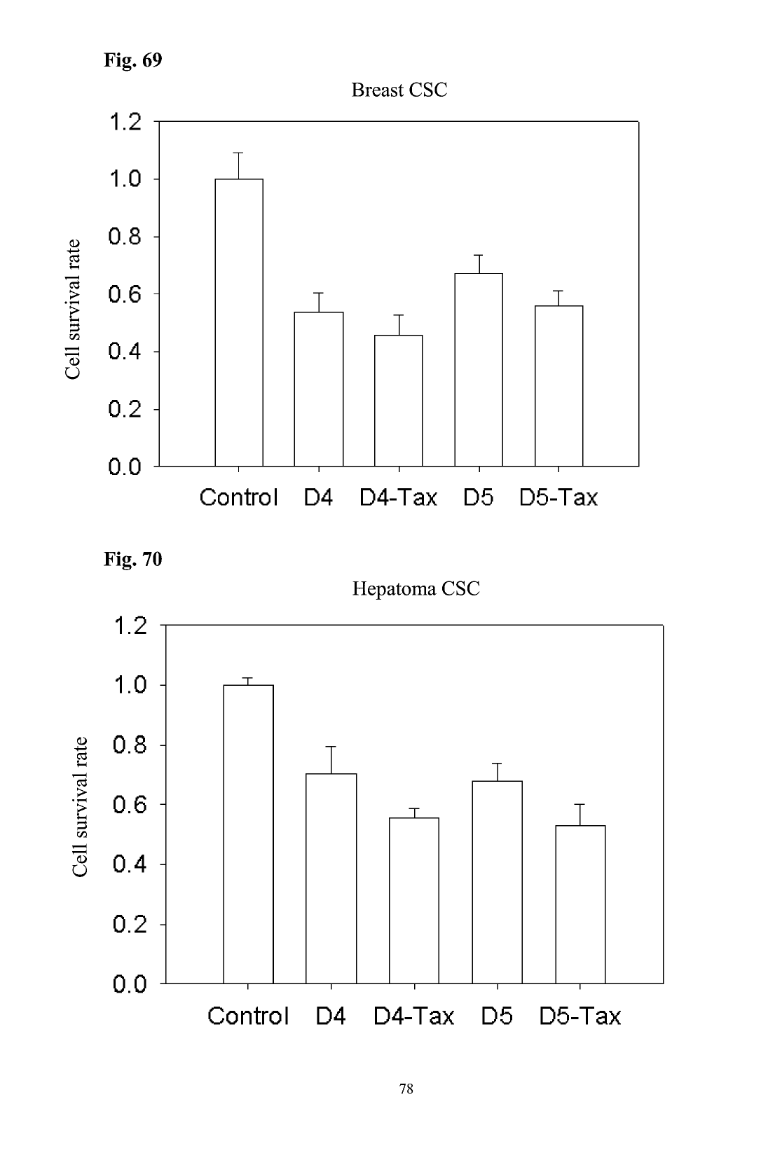

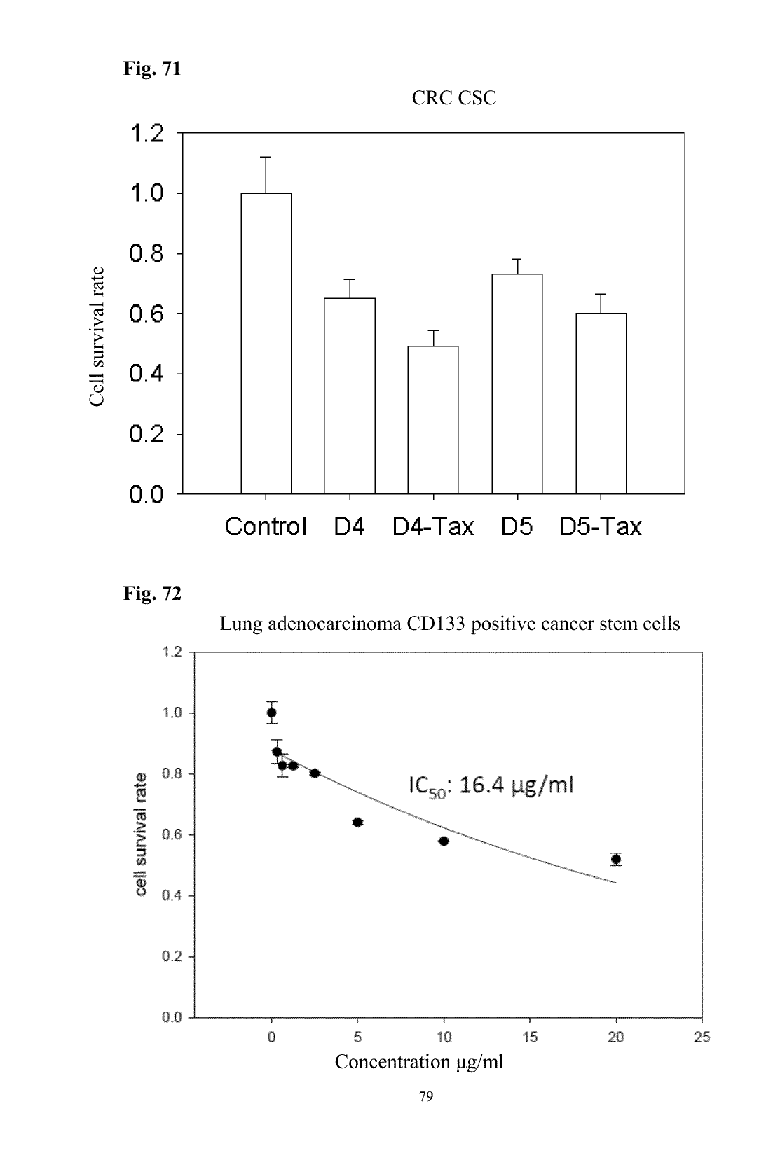

In this embodiment, ethyl acetate extracts of lyophilized powder of A. camphorata concentrate (D4) and ethyl acetate extracts of A. camphorata mycelium (D5) were further tested. Co-administration of the effective A. camphorata extracts with chemotherapy drugs (Cisplatin and Taxol) and co-administration of the effective A. camphorata extracts with ionizing radiation (IR) were tested for anti-cancer effects. The results showed that co-administration of ethyl acetate extracts of lyophilized powder of A. camphorata concentrate (D4) with chemotherapy drugs, Cisplatin, (Table 2 and FIGS. 60-65) or chemotherapy drugs, Taxol, (Table 3 and FIGS. 66-71) partially increased the inhibitory effects on cancer stem cells. Besides, co-administration of ionizing radiation (dosage: 2 Gy or 4 Gy) with ethyl acetate extracts of lyophilized powder of A. camphorata concentrate (D4) partially increased the inhibitory effects on cancer stem cells, D4 had significant inhibitory effects especially with the dosage of 4 Gy of ionizing radiation, (Table 4-6, FIGS. 48-59).

TABLE 2
The half maximal inhibitory concentration (IC₅₀) of co-administration
of A. camphorata extracts with chemotherapy drugs (Cisplatin:
10 μg/ml) for lung cancer stem cells, Glioblastomas multiform
cancer stem cells, Head and neck squamous cell carcinoma cancer
stem cells, colorectal cancer stem cells, breast cancer stem
cells and Hepatoma cancer stem cells.

| | | | | | | Cisplatin |
|---|---|---|---|---|---|---|
| | D1 | D2 | D3 | D4 | D5 | IC₅₀ |
| lung cancer stem | x | x | x | 41.24 | 62.17 | 17.98 |
| cells | | | | μg/ml | μg/ml | μg/ml |
| Glioblastomas | x | x | x | 52.75 | 160.35 | 23.33 |
| multiform cancer | | | | μg/ml | μg/ml | μg/ml |
| stem cells | | | | | | |
| Head and neck | x | x | x | 38.34 | 75.89 | 18.87 |
| squamous cell | | | | μg/ml | μg/ml | μg/ml |
| carcinoma cancer | | | | | | |
| stem cells | | | | | | |
| colorectal cancer | x | x | x | 105.34 | 124.77 | 42.83 |
| stem cells | | | | μg/ml | μg/ml | μg/ml |
| breast cancer stem | x | x | x | 40.05 | 99.43 | 20.23 |
| cells | | | | μg/ml | μg/ml | μg/ml |
| Hepatoma cancer | x | x | x | 106.72 | 145.32 | 40.15 |
| stem cells | | | | μg/ml | μg/ml | μg/ml |

“x”: The maximum drug concentration 400 μg/ml still can not effectively inhibit more than 50% of the cells

TABLE 3
The half maximal inhibitory concentration (IC₅₀) of co-administration
of A. camphorata extracts with chemotherapy drugs (Taxol:
5 ng/ml) for lung cancer stem cells, Glioblastomas multiform
cancer stem cells, Head and neck squamous cell carcinoma cancer
stem cells, colorectal cancer stem cells, breast cancer stem
cells and Hepatoma cancer stem cells.

| | | | | | | Taxol |
|---|---|---|---|---|---|---|
| | D1 | D2 | D3 | D4 | D5 | IC₅₀ |
| lung cancer stem | x | x | x | 30.14 | 50.23 | 13.68 |
| cells | | | | μg/ml | μg/ml | ng/ml |
| Glioblastomas | x | x | x | 43.67 | 105.11 | 20.71 |
| multiform cancer | | | | μg/ml | μg/ml | ng/ml |
| stem cells | | | | | | |
| Head and neck | x | x | x | 29.76 | 60.76 | 10.22 |
| squamous cell | | | | μg/ml | μg/ml | ng/ml |
| carcinoma cancer | | | | | | |
| stem cells | | | | | | |
| colorectal cancer | x | x | x | 88.15 | 107.34 | 31.71 |
| stem cells | | | | μg/ml | μg/ml | ng/ml |
| breast cancer stem | x | x | x | 32.42 | 88.64 | 9.83 |
| cells | | | | μg/ml | μg/ml | ng/ml |
| Hepatoma cancer | x | x | x | 91.33 | 114.23 | 33.45 |
| stem cells | | | | μg/ml | μg/ml | ng/ml |

“x”: The maximum drug concentration 400 μg/ml still can not effectively inhibit more than 50% of the cells

TABLE 4
The half maximal inhibitory concentration (IC₅₀) of co-administration
of A. camphorata extracts with Ionizing Radiation (2 Gy)
for lung cancer stem cells, Glioblastomas multiform cancer
stem cells, Head and neck squamous cell carcinoma cancer
stem cells, colorectal cancer stem cells, breast cancer stem
cells and Hepatoma cancer stem cells.

| | D1 | D2 | D3 | D4 | D5 |
|---|---|---|---|---|---|
| lung cancer stem cells | x | x | x | 51.23 | 68.35 |
| | | | | μg/ml | μg/ml |
| Glioblastomas multiform | x | x | x | 60.73 | 204.45 |
| cancer stem cells | | | | μg/ml | μg/ml |
| Head and neck squamous cell | x | x | x | 40.12 | 73.21 |
| carcinoma cancer stem cells | | | | μg/ml | μg/ml |
| colorectal cancer stem cells | x | x | x | 122.67 | 150.93 |
| | | | | μg/ml | μg/ml |
| breast cancer stem cells | x | x | x | 43.19 | 110.52 |
| | | | | μg/ml | μg/ml |
| Hepatoma cancer stem cells | x | x | x | 137.81 | 196.52 |
| | | | | μg/ml | μg/ml |

“x”: The maximum drug concentration 400 μg/ml still can not effectively inhibit more than 50% of the cells

TABLE 5
The half maximal inhibitory concentration (IC₅₀) of co-administration
of A. camphorata extracts with ionizing radiation (4 Gy)
for lung cancer stem cells, Glioblastomas multiform cancer
stem cells, Head and neck squamous cell carcinoma cancer
stem cells, colorectal cancer stem cells, breast cancer stem
cells and Hepatoma cancer stem cells.

| | D1 | D2 | D3 | D4 | D5 |
|---|---|---|---|---|---|
| lung cancer stem cells | x | x | x | 47.51 | 60.12 |
| | | | | μg/ml | μg/ml |
| Glioblastomas multiform | x | x | x | 50.21 | 196.34 |
| cancer stem cells | | | | μg/ml | μg/ml |
| Head and neck squamous cell | x | x | x | 42.35 | 59.68 |
| carcinoma cancer stem cells | | | | μg/ml | μg/ml |
| colorectal cancer stem cells | x | x | x | 101.45 | 124.13 |
| | | | | μg/ml | μg/ml |
| breast cancer stem cells | x | x | x | 42.69 | 92.47 |
| | | | | μg/ml | μg/ml |
| Hepatoma cancer stem cells | x | x | x | 110.85 | 142.63 |
| | | | | μg/ml | μg/ml |

“x”: The maximum drug concentration 400 μg/ml still can not effectively inhibit more than 50% of the cells

TABLE 6
The half maximal inhibitory concentration (IC₅₀) of the co-administration of A. camphorata
extracts with ionizing radiation (0-4 Gy) for lung cancer stem cells, Glioblastomas multiform
cancer stem cells, Head and neck squamous cell carcinoma cancer stem cells, colorectal cancer
stem cells, breast cancer stem cells and Hepatoma cancer stem cells.

| | D4 (0 Gy) | D4 (2 Gy) | D4 (4 Gy) | D5 (0 Gy) | D5 (2 Gy) | D5(4 Gy) |
|---|---|---|---|---|---|---|
| lung cancer stem | 79.7 | 51.23 | 47.51 | 86.9 | 68.35 | 60.12 |
| cells | | | | | | |
| Glioblastomas | 85.5 | 60.73 | 50.21 | 287.8 | 204.45 | 196.34 |
| multiform cancer | | | | | | |
| stem cells | | | | | | |
| Head and neck | 50.9 | 40.12 | 42.35 | 97.7 | 73.21 | 59.68 |
| squamous cell | | | | | | |
| carcinoma cancer | | | | | | |
| stem cells | | | | | | |
| colorectal cancer | 173.4 | 122.67 | 101.45 | 191.2 | 150.93 | 124.13 |
| stem cells | | | | | | |
| breast cancer stem | 55.8 | 43.19 | 42.69 | 168.6 | 110.52 | 92.47 |
| cells | | | | | | |
| Hepatoma cancer | 167.8 | 137.81 | 110.85 | 268.7 | 196.52 | 142.63 |
| stem cells | | | | | | |

unit: μg/ml

The Inhibitory Effects of 4-acetyl-antroquinonol B Against the Growth of Tumor Cells

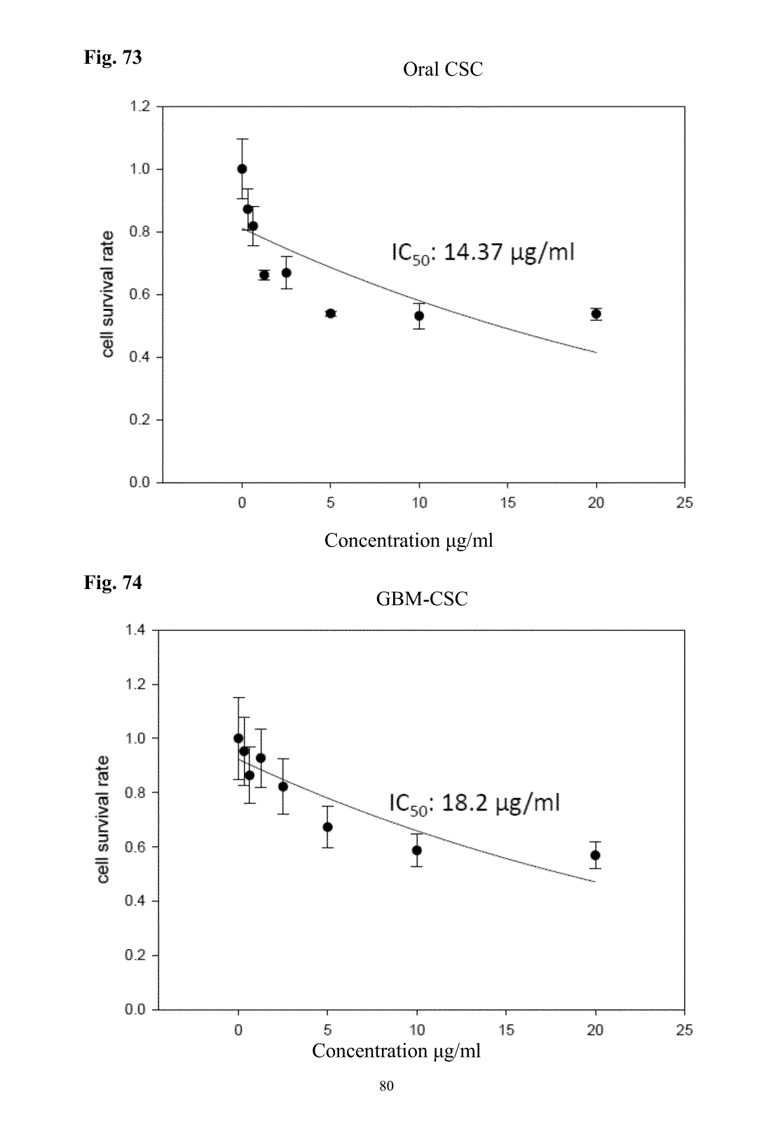

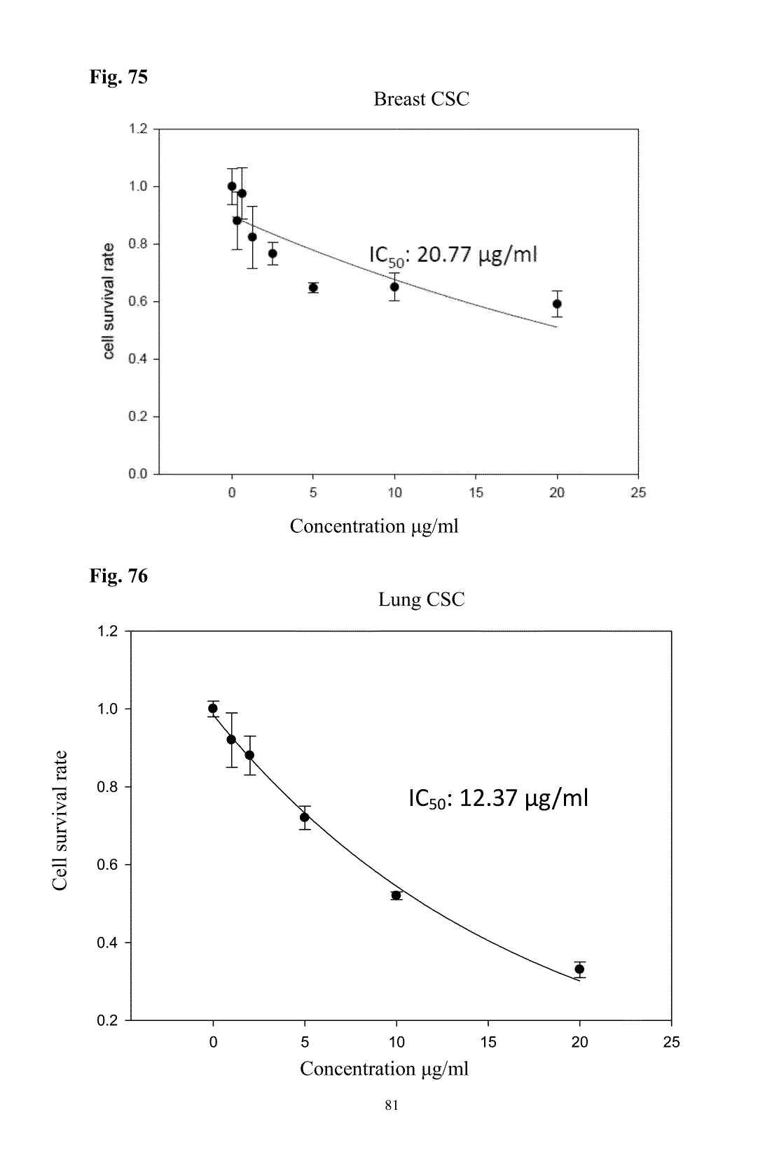

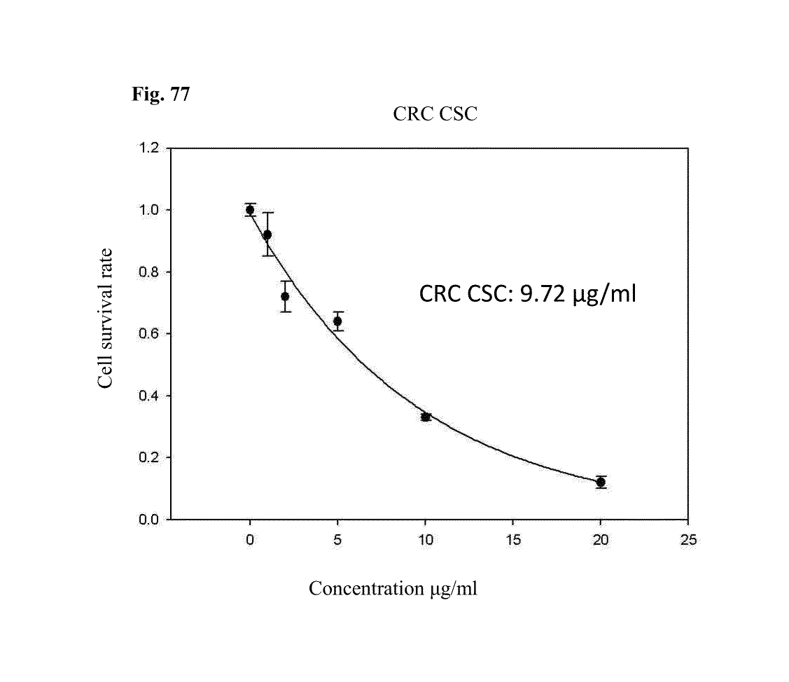

To assess the inhibitory effects of 4-acetyl-antroquinonol B against the growth of cancer stem cells of lung adenocarcinoma CD133 positive tumors, lung cancer, oral cancer, Glioblastomas multiform cancer, breast cancer and colorectal cancer, the cytotoxicity tests for 4-acetyl-antroquinonol B were conducted by MTT assay and they all had effective inhibitory effects on the growth of cancer stem cells, as shown in Table 7 and FIGS. 72-77.

TABLE 7
The half maximal inhibitory concentration (IC50) of 4-acetyl-
antroquinonol B for lung adenocarcinoma CD133 positive cancer
stem cells, oral cancer stem cells, Glioblastomas multiform
cancer stem cells, breast cancer stem cells, lung cancer
stem cells and colorectal cancer stem cells.

| | 4-acetyl-antroquinonol B |
|---|---|
| Lung adenocarcinoma CD133 positive | 16.4 μg/ml |
| cancer stem cells | |
| oral cancer stem cells | 14.37 μg/ml |
| Glioblastomas multiform cancer stem cells | 18.2 μg/ml |
| breast cancer stem cells | 20.77 μg/ml |
| lung cancer stem cells | 12.37 μg/ml |
| colorectal cancer stem cells | 9.72 μg/ml |

### Example 2

### In Vivo treatment of 4-acetyl-antroquinonol B for Tumor Induced by Cancer Stem Cells

### Animal Test:

### Experimental Animals

Immunodeficient mice (NOD/SCID mice about 4-6 weeks old) were purchased from BioLASCO Taiwan Co., Ltd. The test was started after one week domestication.

### Cell Culture

Malignant colorectal cancer DLD1 cancer stem cells were selected for this test. DLD1 cancer stem cell was an adherent cell line possessing a strong metastatic ability. The cell line was cultured in DMEM medium containing 10% fetal bovine serum (FBS), 1% non-essential amino acid (NEAA), and 1% anti-biotic in an incubator with 5% carbon dioxide at 37° C. Subculture was carried out once every three to four days. The cells were treated and suspended in 0.05% trypsin-EDTA for 3-5 minutes, then a serum-containing medium was added to neutralize trypsin. The resultant mixture was centrifuged for 5 minutes (1000 rpm, 20° C.). The supernatant was removed. The cell pellet was gently broken up and an appropriate amount of medium was added. After mixing well, a little cell solution was taken for cell count with a hemacytometer. The cells were diluted to a concentration of 10⁷ cells/ml. Aliquots of 0.15 ml were placed into 1.5 ml microcentrifuge tubes.

Drug Preparation 4-acetyl-antroquinonol B (4-AAQB) was dissolved in DMSO to produce a 4-AAQB solution (250 mg/ml). After completely dissolved, the 4-AAQB solution was dispensed for stock in 4° C. The stock 4-AAQB solution was diluted 500-fold in sterile saline solution to a concentration of 0.5 mg/ml and mixed well for intraperitoneal injection. The dosage of 4-AAQB for mice was 2.5 mg/Kg. The body weight of each mouse was assumed approximately 25 g, thus the injection volume of the diluted 4-AAQB solution was 125 μl. Currently clinical standard chemotherapy drugs Fluorouracil+Oxaliplatin (FOLFOX) were injected at the concentrations of 50 mg/ml and 5 mg/ml, respectively. These two drugs were taken 100 μl each for intravenous injection without dilution, and separate injections were performed to avoid mixing of the two drugs.

### Cancer Stem Cell Injection

One day before injection of colorectal cancer stem cells, 10-fold diluted zoletil 50 and rompun 2% were mixed (1:1), then each mouse was anesthetized by intraperitoneal injection of the mixture (0.25 ml). After each mouse were fully asleep, radiation irradiation was performed to suppress their immune system (radiation dose=0.75 Gy).

The next day, the mice were anesthetized with 2.5% isoflurane, and the hair of the injection site was removed. Before injection, 75% alcohol and povidone-iodine were used to disinfect the injection site. A 29G insulin pen needle was selected for cancer stem cell injection. A tweezer was used to pull the skin of mice, and then the cancer stem cells (10⁶ cells, 0.1 ml) were injected into a subcutaneous place. After injection and confirming no leakage of the cell solution, the mice were moved back into the cage and kept warm to revive. Tumor growth status was continuously observed.

### Tumor Size Measurement

The tumor volume was measured in longest diameter and shortest diameter each week using a caliper. To ensure the accuracy of the measurement, the measurement was carried out by the same person during the experimental period.

Calculation formula of tumor volume: Tumor volume=(a×b²)/2 a=longest diameter; b=shortest diameter.

### Result:

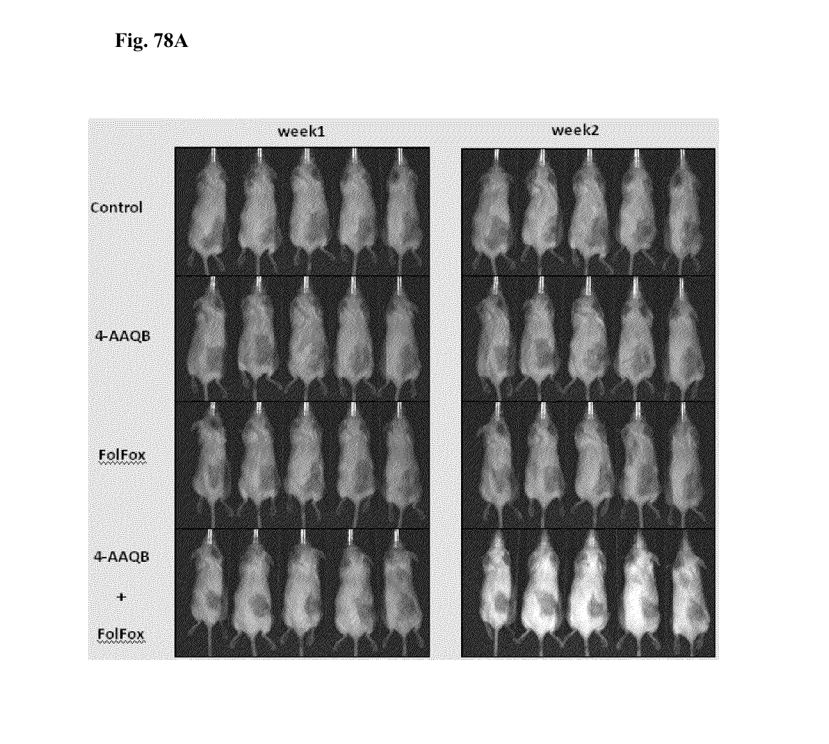

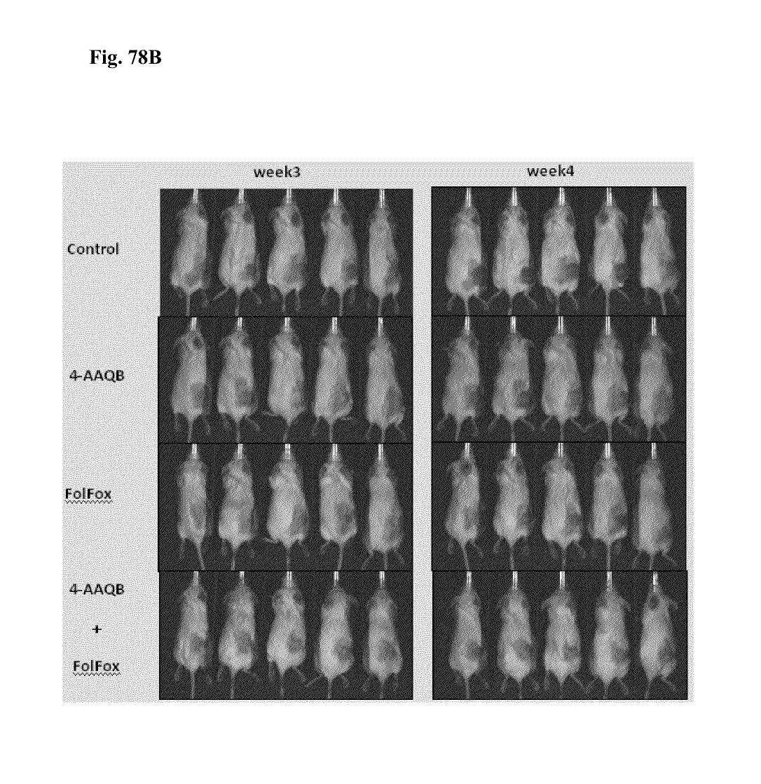

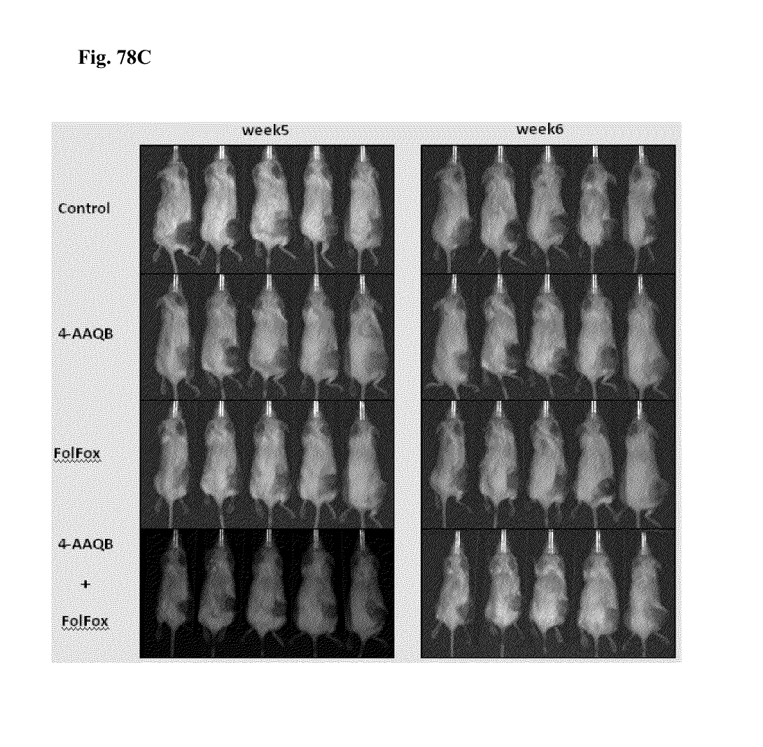

NOD/SCID mice were subcutaneously injected with colorectal cancer stem cells derived from DLD1 cell line to establish xenograft model. Mice were then divided into different groups randomly. One week post tumor injection, treatments commenced. Tumor volumes were measured using a caliper. Photographs were taken on weekly basis for records, as shown in FIG. 78. Each group contains 5 mice. Different treatment regimens were labeled on the left hand column. Four groups were included in this study namely, control, 4-AAQB alone, FolFox alone, and 4-AAQB+FolFox.

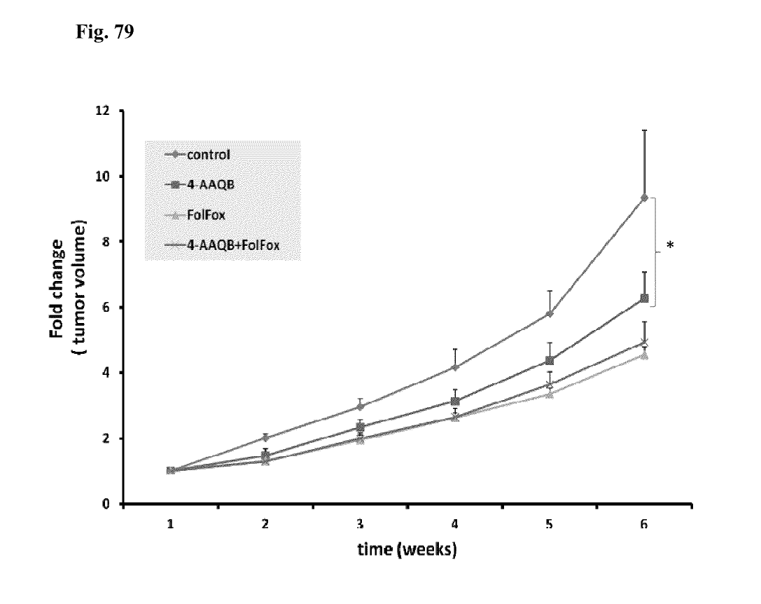

Quantitative analysis of anti-tumor effects by 4-AAQB and other regimens was shown in FIG. 79. The tumor volume of each group was measured in mm³ each week using a caliper. The fold change in tumor volume was plotted against time to demonstrate the difference among the groups. Treatments lasted 5 weeks (starting treatment one week post tumor injection). In FIG. 79, 4-AAQB alone treatment appeared to exert a significant anti-tumorigenesis effect.

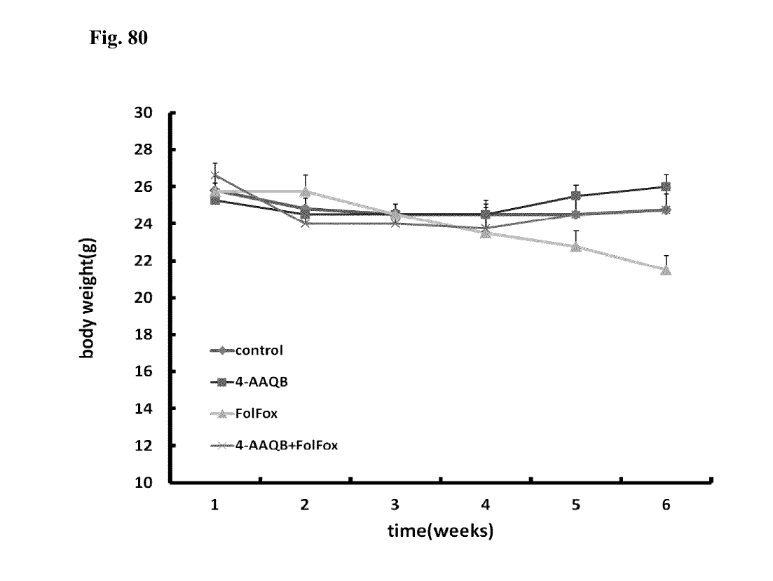

Comparative body weight analysis among different treatment groups was shown in FIG. 80. One of the key landmarks for new drug development was safety. If a drug was toxic to the host, often it would be reflected by the rapid decline in body weight during the experiment. Therefore, the body weight of each treatment group was monitored over the entire experimental period. According to the data shown in FIG. 80, FolFox alone group demonstrated the most decline in body weight by the end of the treatment. More importantly, 4-AAQB and 4-AAQB+FolFox groups did not exhibit significant changes in body weight. This finding implied two important issues. First, 4-AAQB alone at this dosage did not elicit apparent toxicity to the animals. Second, the addition of 4-AAQB in the presence of FolFox might have decreased the side-effect from FolFox treatment. The latter point was important since chemotherapeutic agents used in the clinics often led to severe side-effects in patients and resulted in delay or cessation of the treatment. Thus, this data showed that 4-AAQB had potential to facilitate the patients in completing the treatments without severe side-effects.

What is claimed is:

1. A method for inhibiting tumor growth caused by cancer stem cells in a subject in need thereof, which comprises administering to the subject an effective amount of 4-acetyl-antroquinonol B.

2. The method of claim 1, wherein the cancer stem cells are colorectal cancer stem cells.

3. The method of claim 1, wherein the 4-acetyl-antroquinonol B is administered orally.

4. The method of claim 1, wherein the 4-acetyl-antroquinonol B is administered by injection

5. The method of claim 1, wherein the subject is human.

6. The method of claim 1, wherein the effective amount ranges from 0.1 mg/kg to 10 mg/kg.

7. The method of claim 1, wherein the effective amount ranges from 0.2 mg/kg to 5 mg/kg.

8. The method of claim 1, which comprises administering to the subject a combination of 4-acetyl-antroquinonol B, Fluorouracil, and Oxaliplatin.

9. The method of claim 7, wherein the 4-acetyl-antroquinonol B mitigates body weight loss caused by administration of Fluorouracil and Oxaliplatin.
